(19) United States
(12) Patent Application Publication
(10) Pub. No.: US 2008/0125363 A1
(43) Pub. Date: May 29, 2008

(54) Polymer-Linked Pseudomonas Exotoxin Immunotoxin

(75) Inventors: David Ray Filpula, Piscataway, NJ (US); Karen Yang, Edison, NJ (US); Amartya Basu, Berkeley Heights, NJ (US); Ira H. Pastan, Potomac, MD (US)

Correspondence Address: BOZICEVIC, FIELD & FRANCIS LLP, 1900 UNIVERSITY AVENUE, SUITE 200, EAST PALO ALTO, CA, 94303, US

(73) Assignee: ENZON PHARMACEUTICALS INC., Bridgewater, NJ (US)

(21) Appl. No.: 11/793,165

(22) PCT Filed: Dec. 14, 2005
(86) PCT No.: PCT/US05/45177

§ 371 Date: Nov. 13, 2007

Related U.S. Application Data
(60) Provisional application No. 60/636,007, filed on Dec. 14, 2004.

(51) Int. Cl. A61K 38/00; A61P 35/00; C07K 14/21

(52) U.S. Cl. 514/12; 530/402

(57) ABSTRACT

The present invention relates to polymer conjugates of SS1P, and methods of making using the same.

The present patent application claims the benefit of Provisional Patent Application Ser. No. 60/636,007, filed on Dec. 14, 2004, the disclosure, of which is incorporated herein by reference.

### FIELD OF THE INVENTION

The invention relates to polymer-conjugated immunotoxins targeted to the mesothelin tumor cell antigen. The inventive polymer-conjugated immunotoxins provide a surprisingly enhanced therapeutic index and improved methods of treating tumors and cancers expressing the mesothelin antigen.

### DESCRIPTION OF THE RELATED ART

One in four deaths in the United States are attributed to cancer each year (Jemal et al., 2002; CA Cancer J Clin 52:23-47). While substantial progress has been made in identifying some of the likely environmental and hereditary causes of cancer, the statistics confirm a need for substantial improvement in the therapy for cancers, tumors, and related diseases and disorders.

One of the difficulties in designing successful anticancer therapeutic agents has been the difficulty in selectively targeting tumors and tumor cells while avoiding significant damage to healthy cells and tissues. The advent of monoclonal antibodies, or “mabs,” raised the possibility that a monoclonal antibody that selectively binds to a tumor antigen could be linked to a toxin, in order to provide a safe and selective anticancer immunotoxin therapeutic. Unfortunately, previous attempts have generally not provided satisfactory results, due to a host of technical difficulties. The difficulties included all of the problems associated with protein therapeutics, such as poor tissue penetration, too-rapid renal clearance, and the antigenicity of the protein therapeutic that induced patient immunity to subsequent treatment.

A more sophisticated approach is to create an immunotoxin engineered by linking or recombinantly fusing the active portions of a polypeptide toxin and the active binding domain(s) of a specific targeting antibody. Such engineered immunotoxins provide a reduced molecular weight, relative to those constructed with native mabs, and therefore provide enhanced tissue penetration. Recombinant immunotoxins generally comprise a polypeptide toxin, usually truncated. The polypeptide toxin is linked to, and/or encoded along with, the Fv portion of an antibody or recombinant ligand that serves as the targeting moiety, and that binds specifically to a tumor antigen. A number of such recombinant immunotoxins are now known. The toxin component can be any that is not harmful to non-targeted cells at low concentrations after systemic administration.

One such art-known toxin is the Pseudomonas aeruginosa exotoxin. Native Pseudomonas exotoxin A (“PE”) is an extremely active monomeric protein (molecular weight 66 kD), secreted by Pseudomonas aeruginosa, which inhibits protein synthesis in eukaryotic cells. The native PE sequence is provided in U.S. Pat. No. 5,602,095, incorporated herein by reference. Cytotoxicity is caused by inactivation of the ADP-ribosylation of elongation factor 2 (EF-2). Previous studies with PE have demonstrated that this exotoxin contains three structural domains that act in concert to cause cytotoxicity. Domain Ia (amino acids 1-252) mediates cell binding, and represents a natural targeting mechanism for the toxin. Domain II (amino acids 253-364) is responsible for translocation of the toxin into the cytosol. Domain III (amino acids 400-613) mediates cytotoxicity via ADP ribosylation of elongation factor 2. The function of domain Ib (amino acids 365-399) remains undefined, although a large part of it, amino acids 365-380, can be deleted without loss of cytotoxicity. See Siegall, et al., 1989, J. Biol. Chem. 264: 14256-14261.

Art-known PE based immunotoxins include those in which the Fv portion of an antibody that binds to a tumor-related antigen is fused to a 38 kDa mutant form of PE that has a deletion of its cell binding domain [Pastan, 1997, Biochim. Biophys. Acta. 24: 1333; Kreitman et al. 1994, Blood 83: 426-434; Kreitman et al. 1999, Int. J. Cancer 81: 148-155; Brinkmann et al. 1991, Proc. Natl. Acad. Sci. USA 88: 8616-8620; Reiter et al. 1994, Cancer Res. 54: 2714-2718; Reiter et al. 1994, J. Biol. Chem. 269:18327-18331, all incorporated by reference herein].

Other variations of PE have been tried, including a PE that retains ADP ribosylating activity and the ability to translocate across a cell membrane, but that has a deletion in the receptor binding domain Ia that renders the modified toxin less toxic, as described by U.S. Pat. No. 4,892,872, incorporated by reference herein. U.S. Pat. Nos. 5,696,237, 5,863,745 and 6,051,405, incorporated by reference herein, describe a PE analogous to that of U.S. Pat. No. 4,892,872, that is conjugated to an anti-tumor antigen, exemplified by an anti-Tac antigen.

U.S. Pat. No. 6,809,184, incorporated by reference herein, describes antibodies and antibody fragments that bind to mesothelin, a tumor antigen specific to ovarian cancers, mesotheliomas and several other types of human cancers, as well as recombinant immunotoxins based on fusions of a truncated PE and anti-mesothelin binding proteins.

These previously described immunotoxins, including the anti-mesothelin-PE immunotoxins, are less toxic to mice, allowing higher doses to be given with a substantial increase in antitumor activity. However, liver damage was still a dose limiting problem in the murine model, and this approach does not decrease the immunogenicity of the immunotoxin agent.

One way to enhance the circulating life and reduce the immunogenicity or antigenicity of therapeutic proteins and polypeptides has been to conjugate them to polymers, such as polyalkylene oxides. However, the relatively small size of the polypeptides and their delicate structure/activity relationship, have made polyethylene glycol modification difficult and unpredictable.

To effect covalent attachment of polyalkylene oxides to a protein, the hydroxyl terminals of the polymer must first be converted into reactive functional groups. This process is frequently referred to as “activation” and the product is called “activated PEG” or activated polyalkylene oxide. For example, methoxy poly(ethylene glycol) (mPEG), capped on one end with a functional group, reactive towards amines on a protein molecule, is used in most cases.

A number of activated polymers, such as succinimidyl succinate derivatives of PEG (“SS-PEG”), have been introduced (Abuchowski et al., Cancer Biochem. Biophys. 7:175-186 (1984)). SS-PEG reacts quickly with proteins (30 minutes) under mild conditions yielding active yet extensively modified conjugates. Zalipsky, in U.S. Pat. No. 5,122,614, discloses poly(ethylene glycol)-N-succinimide carbonate and its preparation. This form of the polymer is said to react readily with the amino groups of proteins, as well as low molecular weight peptides and other materials that contain free amino groups. Other linkages between the amino groups of the protein and the PEG are also art known such as urethane linkages (Veronese et al., Appl. Biochem. Biotechnol. 11:141-152 (1985)), carbamate linkages (Beauchamp et al., Analyt. Biochem. 131:25-33 (1983)), and others.

However, despite these and other methods, it has often been found that the resulting conjugates lack sufficient retained activity. For example, Benhar et al. (Bioconjugate Chem. 5:321-326 (1994)) observed that PEGylation of a recombinant single-chain immunotoxin resulted in the loss of specific target immunoreactivity of the immunotoxin. The loss of activity of the immunotoxin was the result of PEG conjugation at two lysine residues within the antibody-combining region of the immunotoxin. To overcome this problem, Benhar et al. replaced these two lysine residues with arginine residues and were able to obtain an active immunotoxin that was 3-fold more resistant to inactivation by derivatization.

Another suggestion for overcoming these problems discussed above is to use longer, higher molecular weight polymers. These materials, however, are difficult to prepare and expensive to use. Further, they provide little improvement over more readily available polymers. Another alternative previously suggested is to attach two strands of polymer via a triazine ring to amino groups of a protein. See, for example, Enzyme 26:49-53 (1981) and Proc. Soc. Exper. Biol. Med., 188:364-369 (1988). However, triazine is a toxic substance that is difficult to reduce to acceptable levels after conjugation. Others have employed releasable polymer conjugates. However, none has heretofore been shown to successfully deliver a tumor killing amount of a PE-based immunotoxin to a targeted tumor.

Thus, there remains a need in the art for a polymer-linked immunotoxin, and particularly for an SE-based immunotoxin that is targeted to the mesothelin tumor antigen, and that avoids or minimizes the disadvantages of previously known SE-based immunotoxins.

### SUMMARY OF THE INVENTION

In order to address these longstanding needs, the invention provides for polymer-conjugates of SS1P, that includes SS1P covalently attached to a substantially non-antigenic polymer. SS1P is preferably a disulfide-linked dimer, the dimer comprising a polypeptide of SEQ ID NO: 5 and a polypeptide of SEQ ID NO: 7.

Optionally, the conjugate is selected so that the SS1P is releasable or nonreleasable e.g., in vivo from the substantially non-antigenic polymer.

Preferably, the substantially non-antigenic polymer ranges in size from about 15 kDa to about 50 kDa.

More preferably, the substantially non-antigenic polymer is a polyalkylene oxide, e.g., polyethylene glycol. Even more preferably, the polymer-conjugated SS1P is selected from one of mPEG-12k-DGA2-RNL8a-SS1P, mPEG-24k-BCN3-SS1P, mPEG-12k-BCN³-mono-SS1P mPEG-12k-RNL8a-SS1P, mono mPEG2-40k-SS1P, mPEG-12k-SC-SS1P mPEG-12k-hydrazide-SS1P, mono mPEG-20k-Ald-SS1P and/or di mPEG-20k-Ald-SS1P.

The number of polymer chains conjugated to the SS1P protein will vary according to the specific application, e.g., ranging from 1 to about 4 polymer chains.

The invention further provides a pharmaceutical composition comprising the inventive polymer-conjugate of SS1P as well as methods of treating a tumor or cancer in an animal comprising administering an effective amount of the polymer-conjugated SS1P to the animal, wherein the tumor or cancer has the property of expressing a mesothelin antigen.

Preferably, the tumor or cancer is a type selected the group of a mesothelioma, an ovarian cancer, a squamous cell carcinoma and/or a pancreatic adenocarcinoma.

Optionally, the pharmaceutical composition also includes one or more additional anti-cancer agent(s). In a method of treatment according to the invention, the method can also include the additional steps of administering one or more additional treatment modalities, such as anti-cancer radiation, anti-cancer agents, and the like, before, after or simultaneously with the administration of the inventive polymer conjugates of SS1P.

### BRIEF DESCRIPTION OF THE DRAWINGS

FIG. 1A illustrates nucleotide sequence 1-2452 of the pPSC7-4 cm plasmid (SEQ ID NO: 1). The open reading frame (“ORF”) encoding SS1V_{L} is from nucleotides 81-401 (SE Q ID NO: 8), and the translated amino acid residues of SS1V_{L} (SEQ ID NO: 7) are also shown below the corresponding codons, the stop site being denoted by an *. The nucleotide sequence of the entire pPSC7-4 cm plasmid (SEQ ID NO: 1) bridges both FIGS. 1A and 1B.

FIG. 1B illustrates nucleotides 2453-3647 of the pPSC7-7 cm plasmid (SEQ ID NO: 1, continued).

FIG. 1C illustrates a restriction map of the pPSC7-7 cm plasmid showing the unique sites.

FIG. 2A illustrates nucleotide sequence 1-1460 of the pPSC7-4 cm plasmid (SEQ ID NO: 2). The ORF encoding the SS1V_{H} protein (SEQ ID NO: 6) bridges both FIGS. 2A and 2B. Translated amino acid residues 1 through 460 of SS1V_{H} are also shown (SEQ ID NO: 5) below the corresponding codons, the stop site being denoted by an *. The entire pPSC7-7 cm plasmid sequence (SEQ ID NO: 2) bridges all of FIGS. 2A-2C.

FIG. 2B illustrates nucleotide sequence 1527 through 4167 of pPSC7-4 cm (SEQ ID NO: 2, continued), and amino acid residues 461 through 472 (SEQ ID NO: 5, continued) of the translated peptide sequence of SS1V_{H} below the corresponding codons. The stop site is denoted by an *.

FIG. 2C illustrates nucleotides 4168 through 4833 of pPSC7-4m (SEQ ID NO: 2, continued).

FIG. 2D illustrates a restriction map of the pPSC7-4 cm plasmid showing unique sites.

FIG. 3 illustrates the immunoreactivity of the exemplified PEG-SS1P conjugates with anti-SS1P antibody that were analyzed by Sandwich ELISA. The results are plotted as absorbance (450 nm) versus concentration (ng). Higher absorbance signifies higher immunoreactivity or antigen—antibody binding. The curves for each tested PEG-SS1P conjugate are identified as follows. “SS1P” represents the unconjugated SS1P protein. This figure signifies that native SS1P elicits a stronger immune response in the host than any of its PEG-conjugated analogues. In other words, PEG-conjugation can conceal the immunogenic epitopes in native SS1P molecule to different degrees. Curves are identified as follows.

| | |
|---|---|
| [symbol] SS1P | [symbol] mPEG-12k-hydrazide-SS1P |
| [symbol] mPEG-24k-BCN3-SS1P | [symbol] mPEG-12k-RNL8a-SS1P |
| [symbol] mPEG-12k-SC-SS1P | [symbol] mPEG-20k-Ald-SS1P |
| [symbol] mPEG-30k-BCN3-mono-SS1P | [symbol] di mPEG-20k-Ald-SS1P |
| [symbol] mPEG2-40k-SS1P | [symbol] mPEG-12k-DGA2-RNL8a- |

FIG. 4A illustrates the structure of a mono mPEG2-40k (a U-PEG with two 20k arms) with a non-releasable linker for conjugating SS1P, and wherein “mPEG” represents,

CH₃—(O—CH₂—CH₂)ₙ—.

FIG. 4B illustrates the structure of mPEG-SC with a non-releasable linker for conjugating SS1P, and “MPEG” is as for FIG. 4A.

FIG. 5A illustrates the structure of an mPEG-DGA2-RNL8a, a releasable linker for conjugating SS1P, and “MPEG” is as for FIG. 4A.

FIG. 5B illustrates the structure of an mPEG-BCN3-U, a releasable linker for conjugating SS1P, and “mPEG” is as for FIG. 4A.

FIG. 5C illustrates the structure of an mPEG-BCN3-mono, a releasable linker for conjugating SS1P, and “MPEG” is as for FIG. 4A.

FIG. 5D illustrates the structure of an mPEG-RNL8a, a releasable linker for conjugating SS1P, and “mPEG” is as for FIG. 4A.

FIG. 6 illustrates the antitumor effect of 24k MPEG BCN3-SS1P on A431-k5 tumors in mice after a single i.v. injection. Control represents no treatment, while SS1P denotes the native, non-conjugated protein. As seen in the figure, PEG-conjugated SS1P can suppress tumor growth for a longer period of time than native SS1P after one dose. The curves are identified as follows.

| | |
|---|---|
| [symbol] 0.5 mg/kg SS1P | [symbol] 3.0 mg/kg |
| [symbol] 1.5 mg/kg | [symbol] 4.0 mg/kg |
| [symbol] 2.0 mg/kg | [symbol] 6.0 mg/kg |
| | [symbol] control |

### DETAILED DESCRIPTION OF THE INVENTION

Accordingly, the present invention provides improved anti-cancer polymer-conjugated immunotoxins. These new polymer-conjugated immunotoxins are capable of treating a number of different types of tumors while reducing or eliminating the above mentioned drawbacks of previously employed immunotoxins. In particular, the present invention provides a polymer-conjugated SE immunotoxin, designated as SS1P, that includes a domain that binds to the mesothelin tumor antigen. The basic strategy is to replace the naturally occurring Domain Ia binding domain with an Fv moiety that targets a tumor specific antigen. In SS1P, the immunotoxin comprises an Fv that binds to mesothelin.

### DEFINITIONS

In order to provide a clear description of the invention, several terms are defined, as follows.

The term, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies), heteroconjugate antibodies (e.g., bispecific antibodies), and recombinant single chain Fv fragments (scFv), disulfide stabilized (dsFv) Fv fragments (See, U.S. Pat. No. 5,747,654, incorporated herein by reference) or pFv fragments. The term “antibody” also includes antigen binding forms of antibodies (e.g., FAb′, F(ab′)₂, Fab, Fv and rIgG. See also, Pierce Catalog and Handbook, 1994-1995 (Pierce Chemical Co., Rockford, Ill.).

An antibody that is immunologically reactive with a particular antigen can be generated by recombinant methods such as selection of libraries of recombinant antibodies in phage or similar vectors. See, e.g., Huse, et al., 1989, Science 246:1275-1281; Ward, et al., 1989, Nature 341:544-546; and Vaughan, et al., 1996, Nature Biotech. 14:309-314.

Typically, an immunoglobulin has a heavy and light chain. Each heavy and light chain contains a constant region and a variable region. Light and heavy chain variable regions contain a “framework” region interrupted by three hypervariable regions, also called complementarity-determining regions or CDRs. The extent of the framework region and CDRs have been defined (see, SEQUENCES OF PROTEINS OF IMMUNOLOGICAL INTEREST, Kabat, E., et al., U.S. Department of Health and Human Services, (1987); which is incorporated herein by reference). The sequences of the framework regions of different light or heavy chains are relatively conserved within a species. The framework region of an antibody, that is the combined framework regions of the constituent light and heavy chains, serves to position and align the CDRs in three dimensional space. The CDRs are primarily responsible for binding to an epitope of an antigen. The CDRs are typically referred to as CDR1, CDR2, and CDR3, numbered sequentially starting from the N-terminus.

The phrase “single chain Fv” or “scFv” refers to an antibody in which the heavy chain and the light chain of a traditional two chain antibody have been joined to form one chain. Typically, a linker peptide is inserted between the two chains to allow for proper folding and creation of an active binding site.

The term “linker peptide” includes reference to a peptide within an antibody binding fragment (e.g., Fv fragment) which serves to indirectly bond the variable heavy chain to the variable light chain.

The phrase “disulfide bond” or “cysteine-cysteine disulfide bond” refers to a covalent interaction between two cysteines in which the sulfur atoms of the cysteines are oxidized to form a disulfide bond. The average bond energy of a disulfide bond is about 60 kcal/mol compared to 1-2 kcal/mol for a hydrogen bond. In the context of this invention, the cysteines which form the disulfide bond are within the framework regions of the single chain antibody and serve to stabilize the conformation of the antibody.

The term, “recombinant” refers to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, under expressed or not expressed at all.

The term “contacting” includes reference to placement in direct physical association. With regards to this invention, the term refers to antibody-antigen binding.

As used herein, “nucleic acid” or “nucleic acid sequence” includes reference to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof as well as conservative variants, i.e., nucleic acids present in wobble positions of codons and variants that, when translated into a protein, result in a conservative substitution of an amino acid.

The term, “encoding” with respect to a specified nucleic acid, includes reference to nucleic acids which comprise the information for translation into the specified protein. The information is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolumn (Proc. Nat'l Acad. Sci. USA 82:2306-2309 (1985), or the ciliate macronucleus, may be used when the nucleic acid is expressed in using the translational machinery of these organisms.

As used herein, the term “anti-mesothelin” in reference to an antibody, includes reference to an antibody that to mesothelin, preferably the binding is selective. In preferred embodiments, the mesothelin is a primate mesothelin such as human mesothelin. In a particularly preferred embodiment, the antibody is generated against human mesothelin synthesized by a non-primate mammal after introduction into the animal of cDNA which encodes human mesothelin.

A “host cell” is a cell which can support the replication or expression of the expression vector. Host cells may be prokaryotic cells such as E. coli, or eukaryotic cells such as yeast, insect, amphibian, or mammalian cells.

The phrase “malignant cell” or “malignancy” refers to tumors or tumor cells that are invasive and/or able to undergo metastasis, i.e., a cancerous cell, or a cancer cell.

The phrase, “mammalian cells” includes reference to cells derived from mammals including humans, rats, mice, guinea pigs, chimpanzees, or macaques. The cells may be cultured in vivo or in vitro.

The term “toxin” preferably refers to highly potent polypeptide-based biological toxins, for example, including abrin, ricin, Pseudomonas exotoxin (PE), diphtheria toxin (DT), botulinum toxin, or modified toxins thereof. For example, PE and DT are highly toxic compounds that typically bring about death through liver toxicity. PE and DT, however, can be modified into a form for use as an immunotoxin by removing the native targeting component of the toxin (e.g., domain Ia of PE and the B chain of DT) and replacing it with a different targeting moiety, such as an antibody, or fragment thereof. In preferred embodiments of the present invention, the toxin is Pseudomonas exotoxin (PE). The term “Pseudomonas exotoxin” or “PE” as used herein refers to a full-length native (naturally occurring) PE or a PE that has been modified. Such modifications may include, but are not limited to, elimination of domain Ia, various amino acid deletions in domains Ib, II and III, single amino acid substitutions and the addition of one or more sequences at the carboxyl terminus as described by Siegall, et al., 1989, J. Biol. Chem. 264:142-56. In a preferred embodiment, the cytotoxic fragment of PE retains at least 50%, preferably 75%, more preferably at least 90%, and most preferably 95% of the cytotoxicity of native PE. In a most preferred embodiment, the cytotoxic fragment is more toxic than native PE.

As used herein, “polypeptide”, “peptide” and “protein” are used interchangeably and include reference to a polymer of amino acid residues. The terms apply to amino acid polymers in which one or more amino acid residue is an artificial chemical analogue of a corresponding naturally occurring amino acid, as well as to naturally occurring amino acid polymers. The terms also apply to polymers containing conservative amino acid substitutions such that the protein remains functional.

The term “residue” or “amino acid residue” or “amino acid” includes reference to an amino acid that is incorporated into a protein, polypeptide, or peptide (collectively “peptide”). The amino acid can be a naturally occurring amino acid and, unless otherwise limited, can encompass known analogs of natural amino acids that can function in a similar manner as naturally occurring amino acids.

Further, the use of singular terms for convenience in description is in no way intended to be so limiting. Thus, for example, reference to a composition comprising a conjugated immunotoxin includes reference to one or more of such conjugates, e.g., including bulk quantities of the conjugates. It is also to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting, since the scope of the present invention will be limited only by the appended claims and equivalents thereof.

Mesothelin, also art-known as CAK1, is a 40 kD GPI-linked glycoprotein antigen present on the surface of mesothelial cells. It is common to ovarian, squamous cell and some stomach cancers, as well as mesotheliomas (Chang, et al., 1992, Cancer Res. 52:181-186; Chang, et al., 1992, J. Surgical Pathology 16:259-268; and Chang, et al., 1996, Nat'l Acad. Sci. USA 93:136-140). It is synthesized as a 69 kD precursor which is then proteolytically processed. The 30 kD amino terminus is secreted and has been termed megakaryocyte potentiating factor (Yamaguchi, et al., 1994, J. Biol. Chem. 269: 805-808). The 40 kD carboxyl terminus remains bound to the membrane as mature mesothelin (Chang, et al., 1996, Nat'l Acad. Sci. USA 93:136-140). Unlike many cell surface antigens present on cancer cells, the membrane-bound form of mesothelin cannot be detected in the blood of cancer patients, and is not shed by cultured cells into medium (Chang, et al., 1992, Cancer Res. 52:181-186). In addition to malignant cells, mesothelin is also found on the cell surface of cells of mesothelial origin, including ovarian cancers. Because damage to cells in these tissues would not lead to life-threatening consequences, the presence of mesothelin on the surface of cancer cells makes it a promising candidate for targeted therapies.

Anti-mesothelin antibodies are elicited, and vectors encoding the mesothelin antigen variable domains, prepared, e.g., as described by U.S. Pat. No. 6,083,502, incorporated herein by reference. There are a number of different art-known configurations for an engineered Fv moiety. One configuration is to encode a recombinant Fv to be expressed as a single-polypeptide chain, with a peptide linker covalently connecting the V_{L} and V_{H} domains, respectively. In the SS1P immunotoxin, the V_{H} domain is encoded as a fusion or chimeric protein, along with the PE toxin domains. The V_{L} domain is expressed by a separate vector, and then disulfide linked to the V_{H} domain of the PE immunotoxin. The resulting construct is referred to in the art as an dsFv.

The following abbreviations may be employed herein in discussing the various linker chemistries for polyethylene glycol conjugates.

| Abbreviation | Name |
|---|---|
| BCN | Bicin |
| DGA | Diglycolic acid |
| RNL | Releasable nitrogen linker |
| Ald | Aldehyde |
| NHS | N-hydroxysuccinimide |
| SC | Succinidyl carbonate |
| Hz | Hydrazide |

### Immunotoxins

PE toxins that may be employed in the present invention as part of engineered immunotoxins include the native PE sequence, cytotoxic fragments of the native sequence, and conservatively modified variants of native PE and its cytotoxic fragments. Cytotoxic fragments of PE include those which are cytotoxic with or without subsequent proteolytic or other processing in the target cell (e.g., as a protein or pre-protein). Cytotoxic fragments of PE include PE40, PE38, and PE35. PE40 is a truncated derivative of PE as previously described in the art. See, Pai, et al, 1991, Proc. Nat'l Acad. Sci. USA 88:3358-62; and Kondo, et al., 1988, J. Bol. Chem. 263: 9470-9475. PE35 is a 35 kD carboxyl-terminal fragment of PE composed of a met at position 280 followed by amino acids 281-364 and 381-613 of native PE. In preferred embodiments, the cytotoxic fragment PE38 is employed. PE38 is a truncated PE pro-protein composed of amino acids 253-364 and 381-613 which is activated to its cytotoxic form upon processing within a cell (see U.S. Pat. No. 5,608,039, incorporated herein by reference).

In one preferred embodiment, PE38 is the toxic moiety of the immunotoxin of this invention, however, other cytotoxic fragments PE35 and PE40 are contemplated and are disclosed in U.S. Pat. Nos. 5,602,095 and 4,892,827, each of which is incorporated herein by reference.

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38.

The term “conservatively modified variants” applies to both amino acid and nucleic acid sequences. With respect to particular nucleic acid sequences, conservatively modified variants refer to those nucleic acid sequences which encode identical or essentially identical amino acid sequences, or if the nucleic acid does not encode an amino acid sequence, to essentially identical nucleic acid sequences. Because of the degeneracy of the genetic code, a large number of functionally identical nucleic acids encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, the artisan will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid.

Pseudomonas exotoxins employed in the invention can be assayed for the desired level of cytotoxicity by assays well known to those of skill in the art. Exemplary toxicity assays are described e.g., by U.S. Pat. No. 6,809,184 at, e.g., Example 2 of that patent, incorporated by reference herein. Thus, cytotoxic fragments of PE and conservatively modified variants of such fragments can be readily assayed for cytotoxicity. A large number of candidate PE molecules can be assayed simultaneously for cytotoxicity by methods well known in the art. For example, subgroups of the candidate molecules can be assayed for cytotoxicity. Positively reacting subgroups of the candidate molecules can be continually subdivided and re-assayed until the desired cytotoxic fragment(s) is identified. Such methods allow rapid screening of large numbers of cytotoxic fragments or conservative variants of PE.

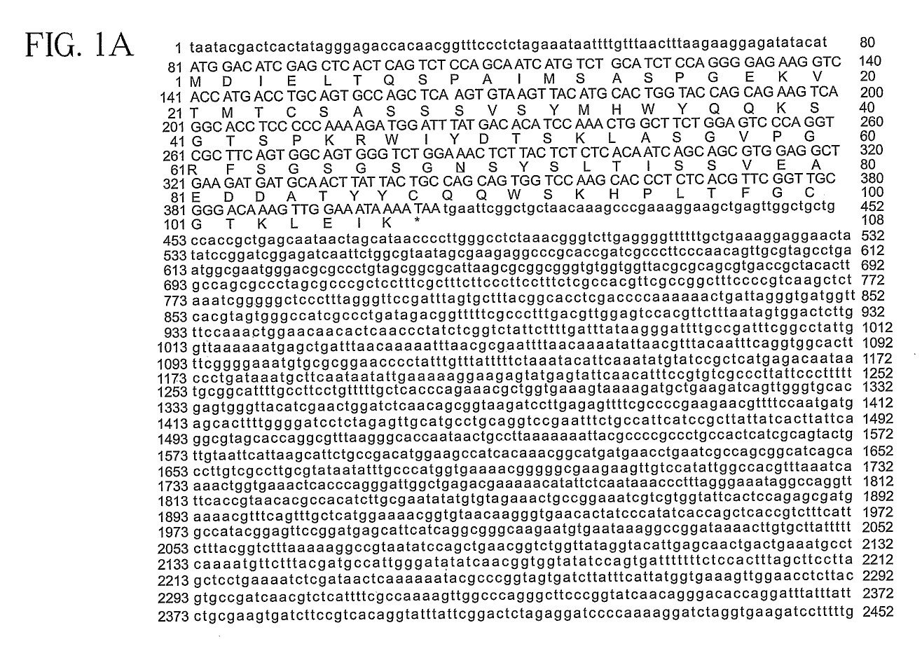

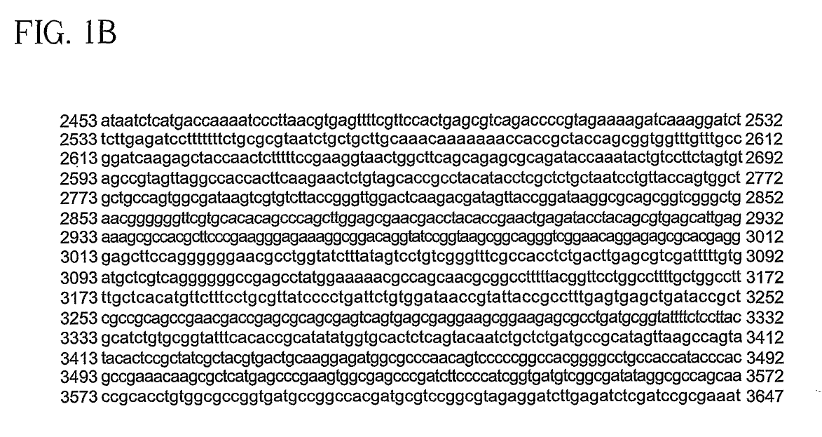

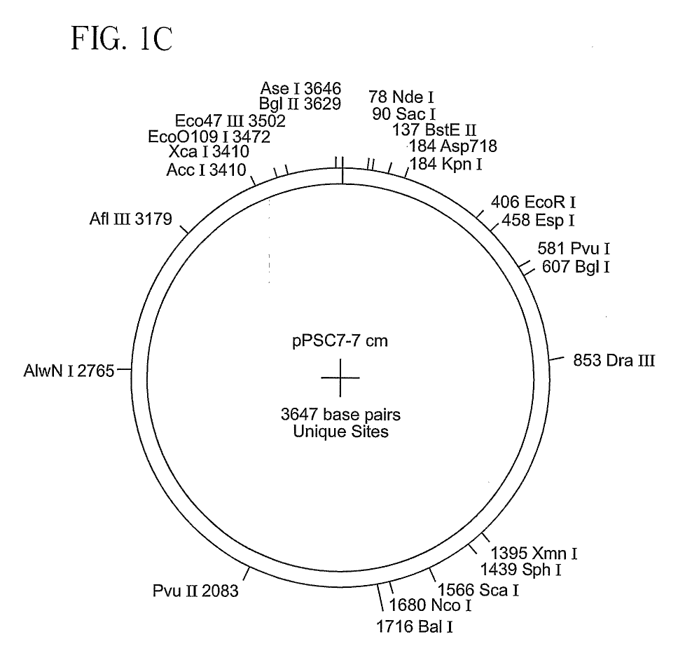

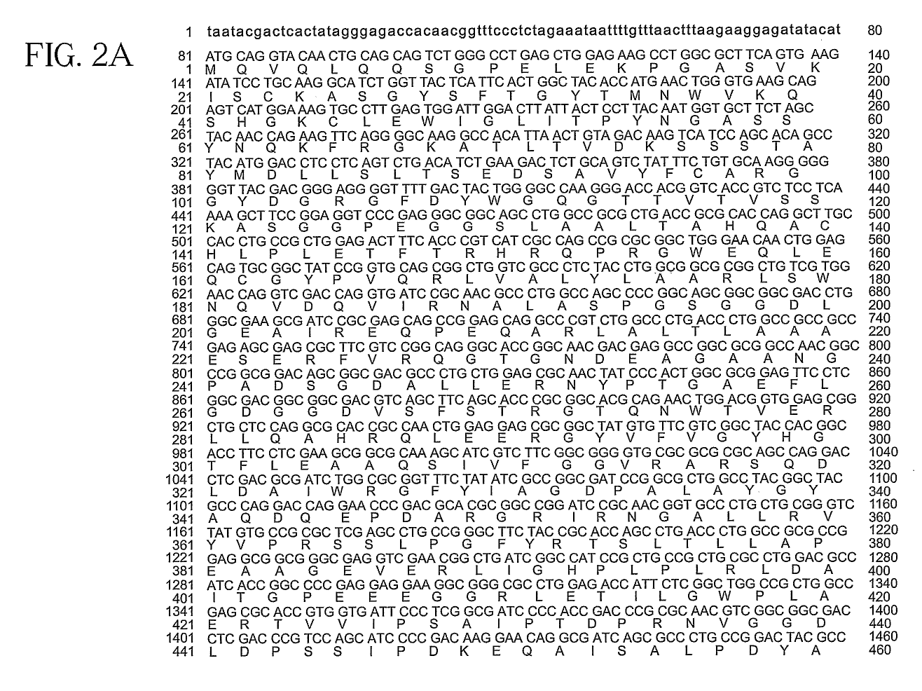

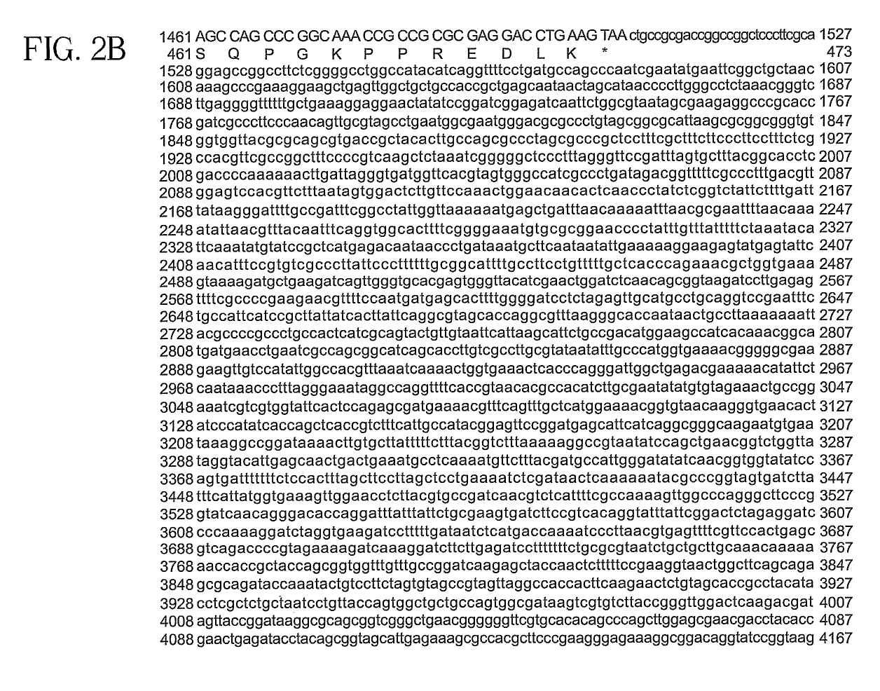

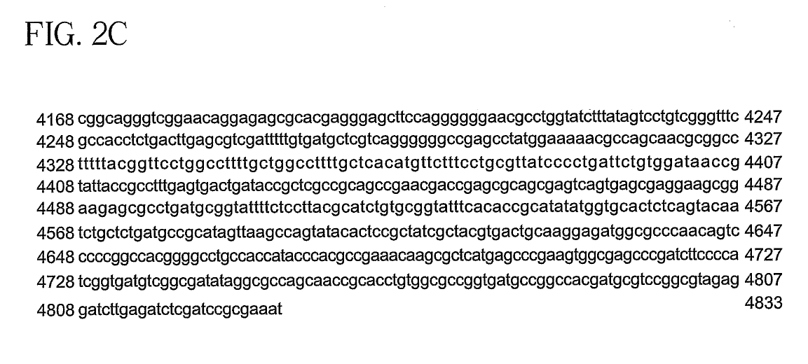

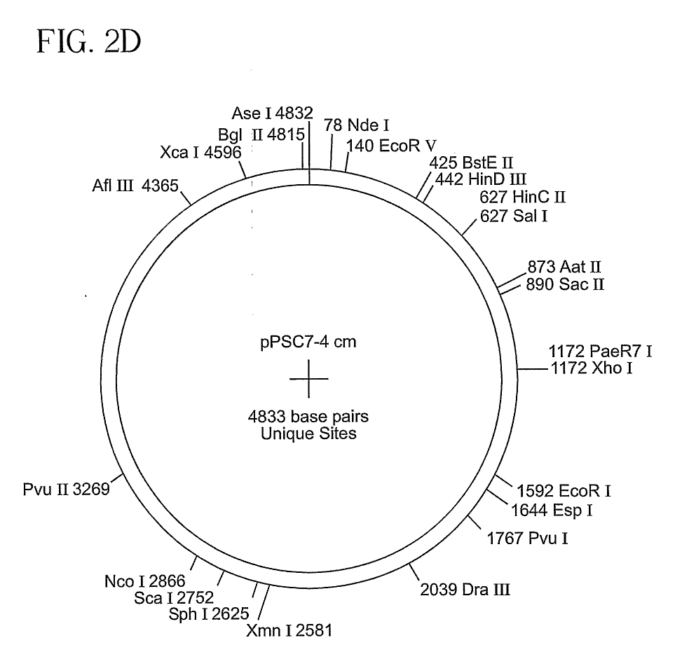

The immunotoxins preferably employed in the conjugates of the invention are generally PE toxins recombinantly modified and fused to an anti-mesothelin Fv moiety. The Fv is preferably a disulfide stabilized or dsFv. The preferred PE immunotoxin is SS1P as described herein, that is an dsFv PE immunotoxin formed by disulfide linkage of an SS1-PE38 V_{H} with an SS1V_{L} peptide. SS1P is a modification of the PE immunotoxin described by FIG. 1 of U.S. Pat. No. 6,809,184 (the '184 patent), incorporated by reference herein. In the '184 patent, the SSV_{L} CDR3 sequence is QQWSGYPLT (SEQ ID NO: 3). This was then modified (mutated) at two positions to give it a higher affinity as described by Chowdhury et al., 1999, Nature Biotechnology 17: 568-572, incorporated by reference herein. Thus, the SS1V_{L} CDR3 sequence of the immunotoxin described herein is QQWSKHPLT (SEQ ID NO: 4). The complete SS1-PE38V_{H} polypeptide sequence is that of SEQ ID NO: 5, that is encoded by the polynucleotide of SEQ ID NO: 6. The complete SS1V_{L} polypeptide sequence is that of SEQ ID NO: 7, that is encoded by the polynucleotide of SEQ ID NO: 8. Thus, admixing the denatured polypeptides having SEQ ID NOs 5 and 7, under refolding conditions, provides SS1P.

### Production of Immunotoxins

Once expressed, the recombinant immunotoxins of the present invention can be purified according to standard procedures of the art, including ammonium sulfate precipitation, affinity columns, column chromatography, and the like (see; generally, R. Scopes, PROTEIN PURIFICATION, Springer—Verlag, N.Y. (1982)). Substantially pure compositions of at least about 90 to 95% homogeneity are preferred, and 98 to 99% or more homogeneity are most preferred for pharmaceutical uses. Purification can be partial, or to homogeneity as desired. If the immunotoxin is to be used therapeutically, the polypeptides should be substantially free of endotoxin.

Methods for expression of single chain antibodies and/or refolding to an appropriate active form, including single chain antibodies, from bacteria such as E. coli have been described and are well-known and are applicable to the immunotoxins employed according to the present invention. See, for example, Buchner, et al., 1992. Anal. Biochem. 205:263-270; Pluckthun, 1991, Biotechnology 9:545; Huse, et al., 1989, Science 246:1275 and Ward, et al., 1989 Nature 341:544, all incorporated by reference herein.

Generally, functional heterologous proteins from E. coli or other bacteria are isolated from inclusion bodies by means of solubilization using strong denaturants, and subsequent refolding. Art-known denaturants include, simply by way of example, urea, potassium thiocyanate, guanadine HCl (“GuHCl”), potassium iodate, and/or sodium iodide and combinations of these. Preferably, GuHCl is employed as a reducing agent, e.g., from about 6 to about 8 M in concentration, under alkaline conditions, e.g., about pH 8. Optionally another reducing agent, dithiothreitol (“DTT”), is employed, either alone or in combination with GuHCl. When DTT is employed, the concentration ranges, simply by way of example, from about 50 mM to about 0.5 mM DTT. During the solubilization step, as is well-known in the art, a reducing agent must be present to separate or denature the disulfide bonds. An exemplary reducing buffer is described hereinbelow: 0.1 M Tris pH 8.0, 6 M guanidine, 2 mM EDTA, and 0.3 M DTE (dithioerythritol).

Renaturation is typically accomplished by dilution (e.g., 100-fold) of the denatured and reduced protein into a refolding buffer, in the presence of an oxidizing agent. Any suitable art-known oxidizing agent can be employed, provided that it allows for correct refolding in good yields. For example, oxidation and refolding can be provided by low molecular weight thiol reagents in reduced and oxidized form, as described in Saxena, et al., 1970, Biochemistry 9: 5015-5021, incorporated by reference herein, and especially as described by Buchner, et al., supra. Renaturation is typically accomplished by dilution (e.g., 100-fold) of the denatured and reduced protein into a refolding buffer. An exemplary refolding buffer is described hereinbelow (Tris HCl 100 mM, pH 10.0, 25 mM EDTA, NaCl 0.1 M, GSSG 551 mg/L, 0.5 M Arginine). GSSG is the oxidized form of glutathione.

As a modification to the two chain antibody purification protocol, the heavy and light chain regions are separately solubilized and reduced and then combined in the refolding solution. A preferred yield is obtained when these two proteins are mixed in a molar ratio such that a 5-fold molar excess of one protein over the other is not exceeded. It is desirable to add excess oxidized glutathione or other oxidizing low molecular weight compounds to the refolding solution after the redox-shuffling is completed.

### Polymer-Immunotoxin Conjugates

The immunotoxin-polymer conjugates of the present invention generally correspond to formula (I):

(R₁)_{z}—NH—(ITX) (I)

wherein

### (ITX) represents the immunotoxin, or a derivative or fragment thereof;

### NH— is an amino group of an amino acid found on the ITX, derivative or fragment thereof for attachment to the polymer;

z is a positive integer, preferably from about 1 to about 6; and

### R₁ is a substantially non-antigenic polymer residue that is attached to the ITX in a releasable or non-releasable form.

The non-antigenic polymer residue portion of the conjugate (R₁) can be selected from among a non-limiting list of polymer based systems such as:

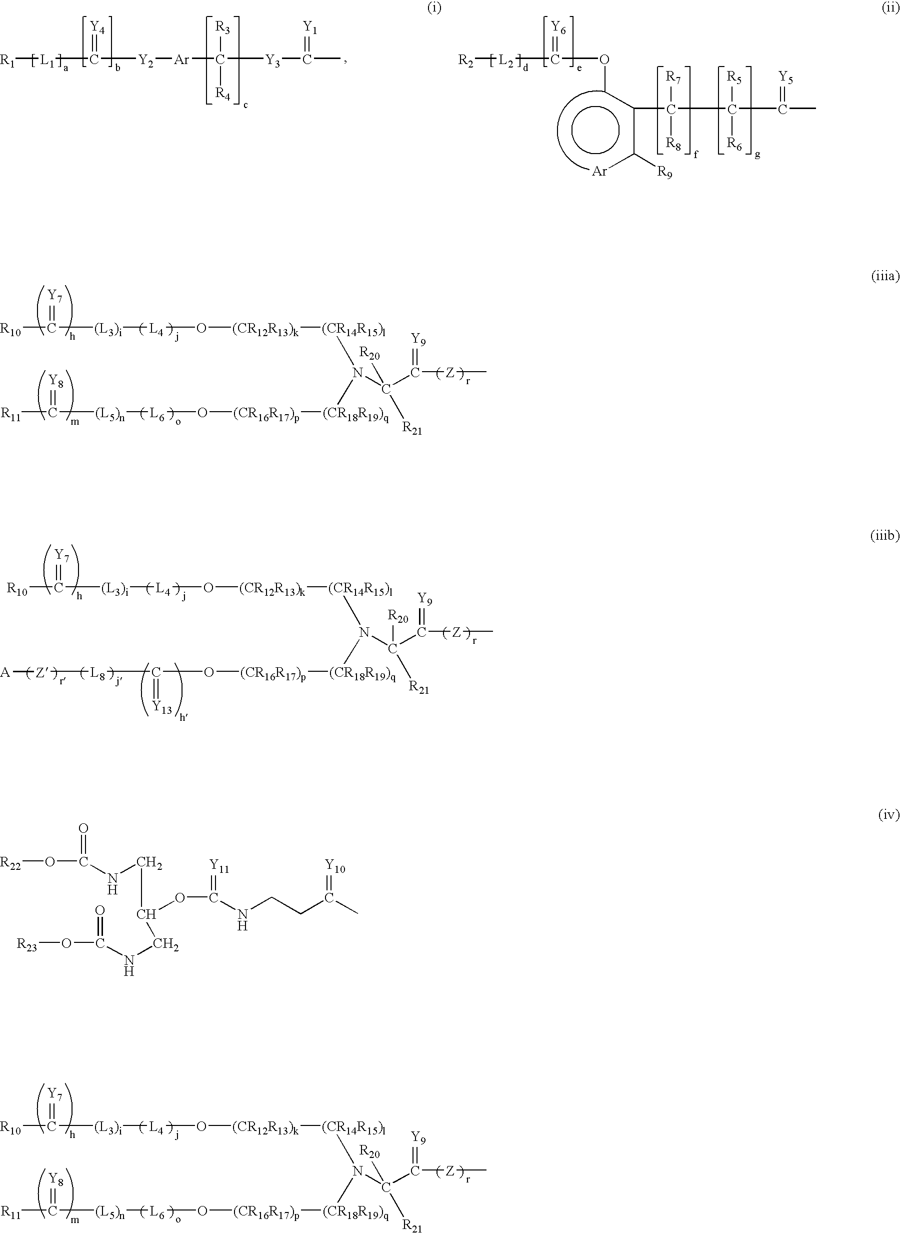

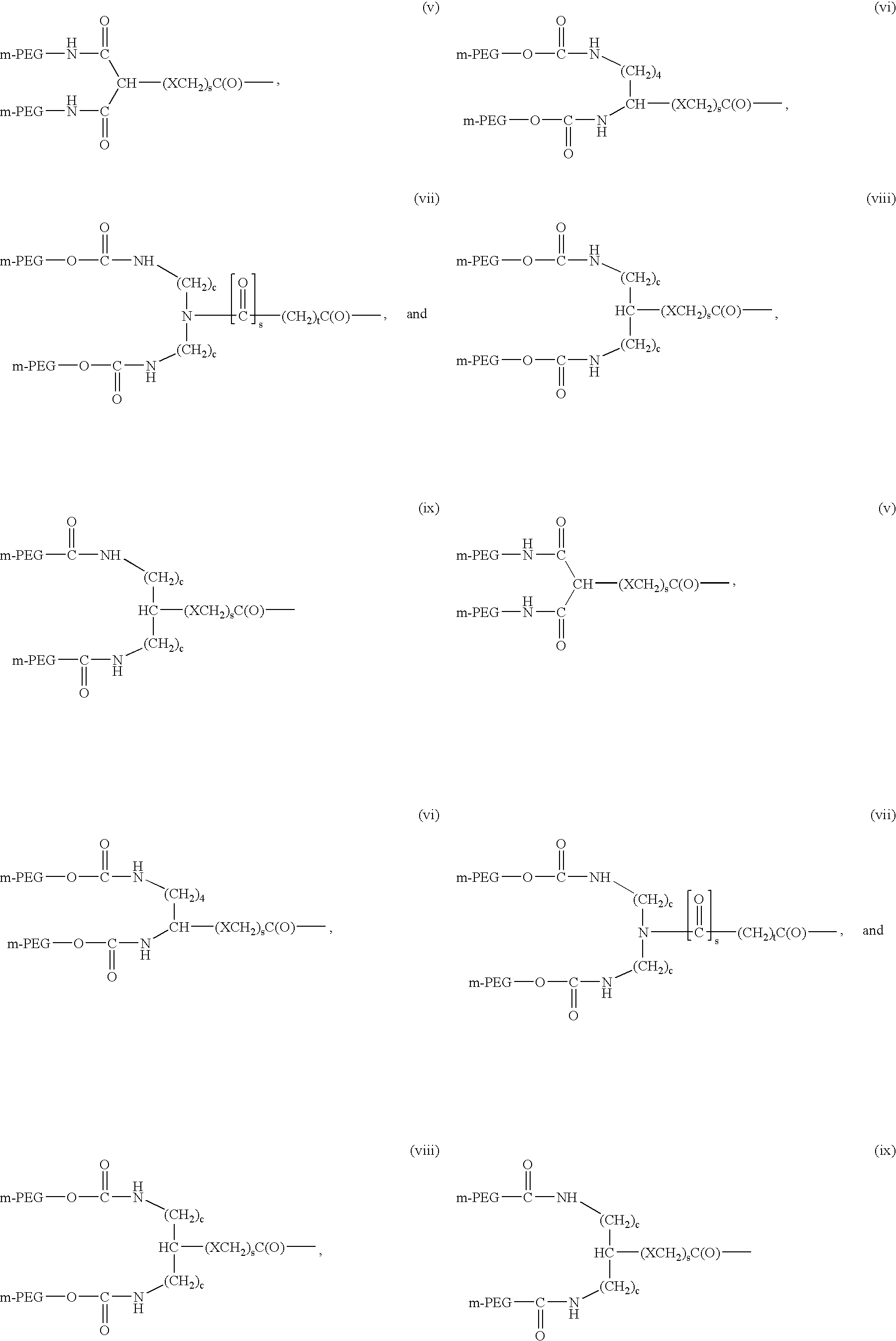

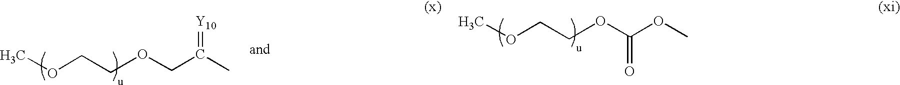

wherein:

R₁₋₂, R₁₀₋₁₁, and R₂₂₋₂₃ may be the same or different and are independently selected non-antigenic polymer residues;

R₃₋₉, R₁₂₋₂₁ and R₂₄ (see below) are the same or different and are each independently selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxys;

Ar is a moiety which forms a multi-substituted aromatic hydrocarbon or a multi-substituted heterocyclic group;

Y₁₋₁₁ and Y₁₃ may be the same or different and are independently selected from O, S and NR₂₄;

A is selected from among alkyl groups, targeting moieties, leaving groups, functional groups, diagnostic agents, and biologically active moieties;

X is O, NQ, S, SO or SO₂; where Q is H, C₁₋₈ alkyl, C₁₋₈ branched alkyl, C₁₋₈ substituted alkyl, aryl or aralkyl;

Z and Z′ are independently selected from among moieties actively transported into a target cell, hydrophobic moieties, bifunctional linking moieties and combinations thereof;

L₁₋₆ and L₈ may be the same or different and are independently selected bifunctional linker groups;

a, c, d, f, g, i, j, j′, k, l, n, o, p and t may be the same or different and are independently 0 or a positive integer, preferably, in most aspects;

b, r, r′, s, h, h′ and m may be the same or different and are independently 0 or 1;

mPEG is H₃CO(—CH₂CH₂O)ᵤ— and

u is a positive integer, preferably from about 10 to about 2,300, and more preferably from about 200 to about 1000.

Within the above, it is preferred that Y₁₋₁₁ and Y₁₃ are O; R₃₋₈, R₁₂₋₂₁ and R₂₄ are each independently either hydrogen or C₁₋₆ alkyls, with methyl and ethyl being the most preferred alkyls and R₉ is preferably CH₃.

In a further aspect of the invention, the polymer portion of the conjugate can be one which affords multiple points of attachment for the immunotoxin. A non-limiting list of such systems include:

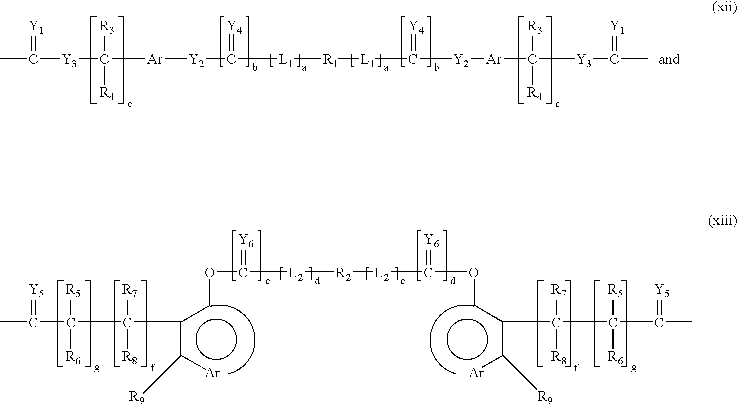

wherein all variables are the same as that set forth above.

The activated polymers which can be employed to make the immunotoxin conjugates will naturally correspond directly with the polymer portions described above. The chief difference is the presence of a leaving or activating group, sometimes designated herein as B₁, which facilitates the releasable attachment of the polymer system to an amine group found on the immunotoxin. Thus, compounds (i)-(xiii) include a leaving or activating group such as:

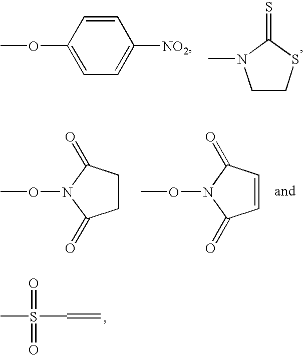

or other suitable leaving or activating groups such as N-hydroxysuccinimidyl, N-hydroxybenzotriazolyl, halogen, N-hydroxyphthalimidyl, p-nitrophenoxy, imidazolyl, thiazolidinyl thione, O-acyl ureas, pentafluorophenol or 2,4,6-tri-chlorophenol or other suitable leaving groups apparent to those of ordinary skill, found in the place where the immunotoxin attaches after the conjugation reaction.

For purposes of the present invention, leaving groups are to be understood as those groups which are capable of reacting with an amine group (nucleophile) found on an immunotoxin, e.g. on a Lys.

For purposes of the present invention, the foregoing are also referred to as activated polymer linkers. The polymer residues are preferably polyalkylene oxide-based and more preferably polyethylene glycol (PEG) based wherein the PEG is either linear or branched.

Referring now to the activated polymers described above, it can be seen that the Ar is a moiety which forms a multi-substituted aromatic hydrocarbon or a multi-substituted heterocyclic group. A key feature is that the Ar moiety is aromatic in nature. Generally, to be aromatic, the π electrons must be shared within a “cloud” both above and below the plane of a cyclic molecule. Furthermore, the number of π electrons must satisfy the Huckle rule (4n+2). Those of ordinary skill will realize that a myriad of moieties will satisfy the aromatic requirement of the moiety and thus are suitable for use herein with halogen(s) and/or side chains as those terms are commonly understood in the art.

In some preferred aspects of the invention, the activated polymer linkers are prepared in accordance with commonly-assigned U.S. Pat. Nos. 6,180,095, 5,965,119 and 6,303,569, the contents of which are incorporated herein by reference. Within this context, the following activated polymer linkers are preferred:

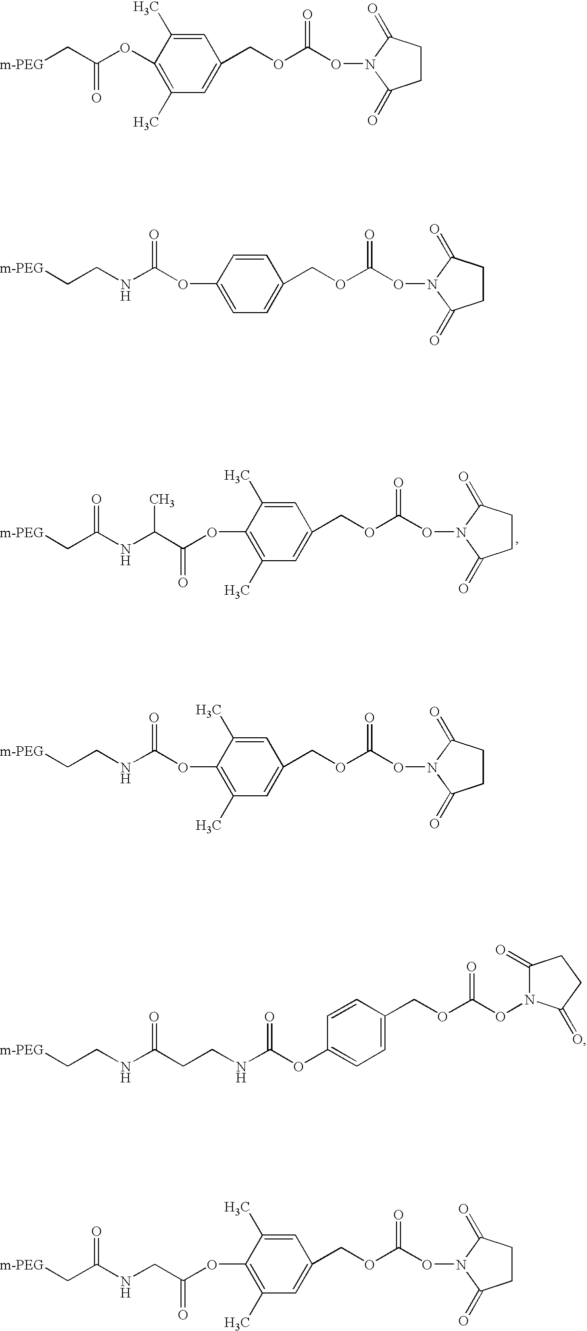

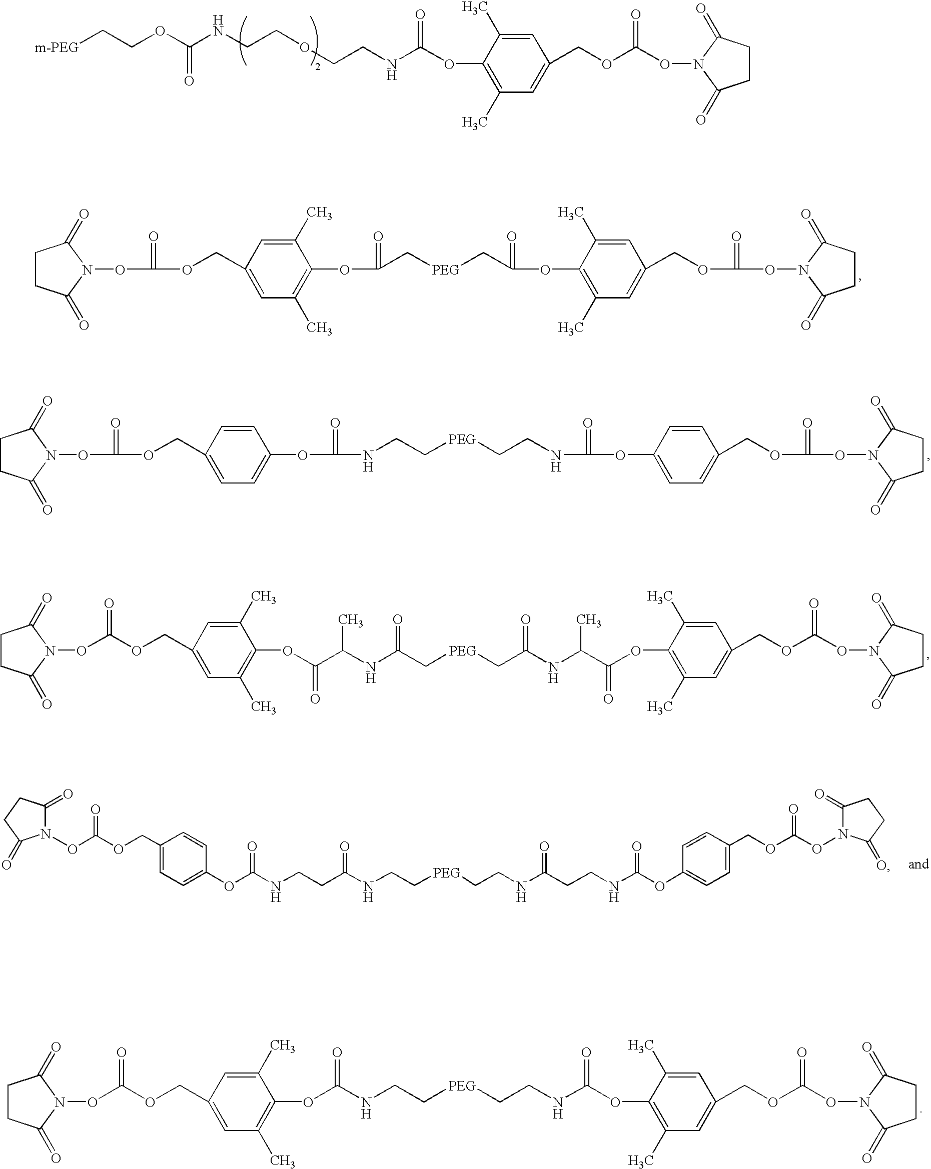

In one alternative aspect of the invention, the immunotoxin polymer conjugates are made using certain branched or bicine polymer residues such as those described in commonly assigned U.S. patent application Ser. Nos. 10/218,167, 10/449,849 and 11/011,818. The disclosure of each such patent application is incorporated herein by reference. A few of the preferred activated polymers include:

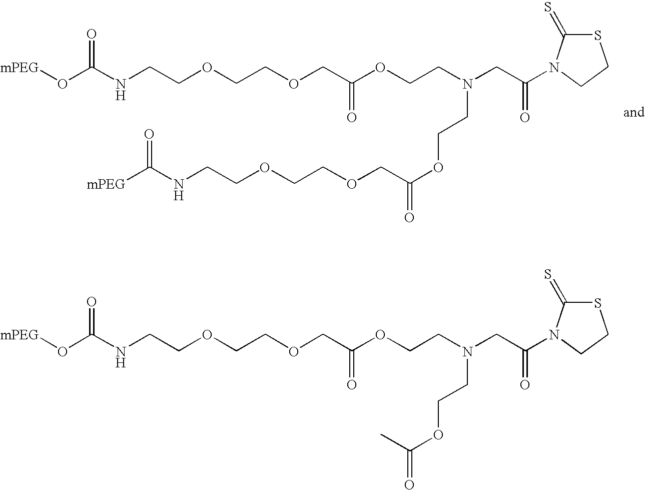

### It should also be understood that the leaving group shown above is only one of the suitable groups and the others mentioned herein can also be used without undue experimentation.

In alternative aspects, the activated polymer linkers are prepared using branched polymer residues such as those described commonly assigned U.S. Pat. Nos. 5,643,575; 5,919,455 and 6,113,906, the disclosure of each being incorporated herein by reference. Such activated polymers correspond to polymer systems (v)-(ix) with the following being representative:

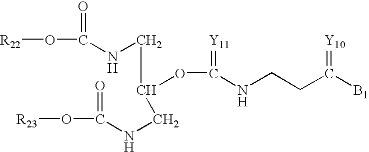

wherein all variables are as previously defined.

### Substantially Non-Antigenic Polymers

As stated above, R₁₋₂, R₁₀₋₁₁ and R₂₂₋₂₃ are preferably each water soluble polymer residues which are preferably substantially non-antigenic such as polyalkylene oxides (PAO's) and more preferably polyethylene glycols such as mPEG. For purposes of illustration and not limitation, the polyethylene glycol (PEG) residue portion of R₁₋₂, R₁₀₋₁₁, and R₂₂₋₂₃ can be selected from among:

J-O—(CH₂CH₂O)ᵤ—

J-O—(CH₂CH₂O)ᵤ—CH₂C(O)—O—,

J-O—(CH₂CH₂O)ᵤ—CH₂CH₂NR₂₅—, and

J-O—(CH₂CH₂O)ᵤ—CH₂CH₂SH—,

wherein:

u is the degree of polymerization, i.e. from about 10 to about 2,300;

R₂₅ is selected from among hydrogen, C₁₋₆ alkyls, C₂₋₆ alkenyls, C₂₋₆ alkynyls, C₃₋₁₂ branched alkyls, C₃₋₈ cycloalkyls, C₁₋₆ substituted alkyls, C₂₋₆ substituted alkenyls, C₂₋₆ substituted alkynyls, C₃₋₈ substituted cycloalkyls, aryls substituted aryls, aralkyls, C₁₋₆ heteroalkyls, substituted C₁₋₆ heteroalkyls, C₁₋₆ alkoxy, phenoxy and C₁₋₆ heteroalkoxy, and

J is a capping group, i.e. a group which is found on the terminal of the polymer and, in some aspects, can be selected from any of NH₂, OH, SH, CO₂H, C₁₋₆ alkyls, preferably methyl, or other PEG terminal activating groups, as such groups are understood by those of ordinary skill.

Preferred J groups used for polymer capping include moieties such as OH, NH₂, SH, CO₂H, C₁₋₆ alkyl moieties, such as CH₃,

In one particularly preferred embodiment, R₁₋₂, R₁₀₋₁₁ and R₂₂₋₂₃ are selected from among,

CH₃—O—(CH₂CH₂O)ᵤ—,CH₃—O—(CH₂CH₂O)ᵤ—CH₂C(O)—O—,

CH₃—O—(CH₂CH₂O)ᵤ—CH2CH NH— and CH₃—O—(CH₂CH₂O)ᵤ—CH2CH₂ SH—,

where u is a positive integer, preferably selected so that the weight average molecular weight from about 200 to about 80,000 Da. More preferably, R₁₋₂, R₁₀₋₁₁, and R₂₂₋₂₃ independently have a weight average molecular weight of from about 2,000 Da to about 42,000 Da, with a weight average molecular weight of from about 5,000 Da to about 40,000 Da being most preferred. Other molecular weights are also contemplated so as to accommodate the needs of the artisan.

PEG is generally represented by the structure:

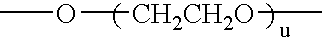

and R₁₋₂, R₁₀₋₁₁ and R₂₂₋₂₃ preferably comprise residues of this formula. The degree of polymerization for the polymer represents the number of repeating units in the polymer chain and is dependent on the molecular weight of the polymer.

Also useful are polypropylene glycols, branched PEG derivatives such as those described in commonly-assigned U.S. Pat. No. 5,643,575 (the '575 patent), “star-PEG's” and multi-armed PEG's such as those described in Shearwater Corporation's 2001 catalog “Polyethylene Glycol and Derivatives for Biomedical Application”. The disclosure of each of the foregoing is incorporated herein by reference. The branching afforded by the '575 patent allows secondary or tertiary branching as a way of increasing polymer loading on a biologically active molecule from a single point of attachment. It will be understood that the water-soluble polymer can be functionalized for attachment to the bifunctional linkage groups if required without undue experimentation.

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In a further embodiment, and as an alternative to PAO-based polymers, R₁₋₂, R₁₀₋₁₁, and R₂₂₋₂₃ are each optionally selected from among one or more effectively non-antigenic materials such as dextran, polyvinyl alcohols, carbohydrate-based polymers, hydroxypropylmeth-acrylamide (HPMA), polyalkylene oxides, and/or copolymers thereof. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated and that other polyalkylene oxide derivatives such as the polypropylene glycols, etc. are also contemplated.

### Bifunctional Linker Groups:

In many aspects of the invention, L₁₋₆ and L₈ are linking groups which facilitate attachment of the polymer strands, e.g. R₁₋₂, R₁₀₋₁₁, and/or R₂₂₋₂₃. The linkage provided can be either direct or through further coupling groups known to those of ordinary skill. In this aspect of the invention, L₁₋₆ and L₈ may be the same or different and can be selected from a wide variety of groups well known to those of ordinary skill such as bifunctional and heterobifunctional aliphatic and aromatic-aliphatic groups, amino acids, etc. Thus, L₁₋₆ and L₈ can be the same or different and include groups such as:

—NH(CH₂CH₂)₂O—

—NH(CH₂CH₂)(CH₂CH₂O)NH—

—O(CH₂CH₂)NH—

—O(CH₂CH₂)O—

—NH(CH₂CH₂)NH—

—NH(CH₂CH₂)(CH₂CH₂O)—

—NH(CH₂CH₂O)—

—NH(CH₂CH₂O)(CH₂)NH—

—NH(CH₂CH₂O)₂—

—O(CH₂)₃NH—

—O(CH₂)₃O—

—O(CH₂CH₂O)₂NH—

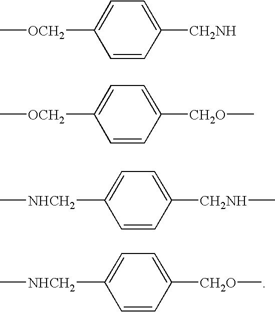

Preferably, L₁₋₆ and L₈ are selected from among:

—C(O)CH₂OCH₂C(O)—;

—C(O)CH₂NHCH₂C(O)—;

—C(O)CH₂SCH₂C(O)—;

—C(O)CH₂CH₂CH₂C(O)—, and

—C(O)CH₂CH₂C(O)—.

Alternatively, suitable amino acid residues can be selected from any of the known naturally-occurring L-amino acids is, e.g., alanine, valine, leucine, etc. and/or a combination thereof, to name but a few. L₁₋₆ and L₈ can also include a peptide which ranges in size, for instance, from about 2 to about 10 amino acid residues.

Derivatives and analogs of the naturally occurring amino acids, as well as various art-known non-naturally occurring amino acids (D or L), hydrophobic or non-hydrophobic, are also contemplated to be within the scope of the invention.

### A Moieties

1. Leaving or Activating Groups

In those aspects where A is an activating group, suitable moieties include, without limitation, groups such as N-hydroxybenzotriazolyl, halogen, N-hydroxyphthalimidyl, p-nitrophenoxyl, imidazolyl, N-hydroxysuccinimidyl; thiazolidinyl thione, O-acyl ureas, pentafluorophenoxyl, 2,4,6-trichlorophenoxyl or other suitable leaving groups that will be apparent to those of ordinary skill.

For purposes of the present invention, leaving groups are to be understood as those groups which are capable of reacting with a nucleophile found on the desired target, i.e. a biologically active moiety, a diagnostic agent, a targeting moiety, a bifunctional spacer, intermediate, etc. The targets thus contain a group for displacement, such as NH₂ groups found on proteins, peptides, enzymes, naturally or chemically synthesized therapeutic molecules such as doxorubicin, spacers such as mono-protected diamines. It is to be understood that those moieties selected for A can also react with other moieties besides biologically active nucleophiles.

2. Functional Groups

A can also be a functional group. Non-limiting examples of such functional groups include maleimidyl, vinyl, residues of sulfone, hydroxy, amino, carboxy, mercapto, hydrazide, carbazate and the like which can be attached to the bicine portion through an amine-containing spacer. Once attached to the bicine portion, the functional group, (e.g. maleimide), can be used to attach the bicine-polymer to a target such as the cysteine residue of a polypeptide, amino acid or peptide spacer, etc.

3. Alkyl Groups

In those aspects of formula (I) where A is an alkyl group, a non-limiting list of suitable groups consists of C₁₋₆ alkyls, C₂₋₆ alkenyls, C₂₋₆ alkynyls, C₃₋₁₉ branched alkyls, C₃₋₈ cycloalkyls, C₁₋₆ substituted alkyls, C₂₋₆ substituted alkenyls, C₂₋₆ substituted alkynyls, C₃₋₈ substituted cycloalkyls, aralkyls, C₁₋₆ heteroalkyls, and substituted C₁₋₆ heteroalkyls.

### Z Moieties and their Function

In one aspect of the invention Z and Z′ are L₇—C(═Y₁₂) wherein L₇ is a bifunctional linker selected from among the group which defines L₁₋₆, and Y₁₂ is selected from among the same groups as that which defines Y₁. In this aspect of the invention, the Z group serves as the linkage between the immunotoxin and the remainder of the polymer delivery system. In other aspects of the invention, Z is a moiety that is actively transported into a target cell, a hydrophobic moiety, and combinations thereof. The Z′ when present can serve as a bifunctional linker, a moiety that is actively transported into a target cell, a hydrophobic moiety, and combinations thereof.

In this aspect of the invention, the releasable polymer systems are prepared so that in vivo hydrolysis cleaves the polymer from the immunotoxin and releases the immunotoxin into the extracellular fluid, while still linked to the Z moiety. For example, one potential Z-B combination is leucine-immunotoxin.

### Preparation of SS1P Conjugates

Synthesis of specific protein-polymer conjugates or prodrugs is set forth in the Examples. For purposes of illustration, however, suitable conjugation reactions include reacting SS1P immunotoxin or fragment, etc. with a suitably activated polymer system described herein. The reaction is preferably carried out using conditions well known to those of ordinary skill for protein modification, including the use of a PBS buffered system, etc. with the pH in the range of about 6.5-8.5. It is contemplated that in most instances, an excess of the activated polymer will be reacted with the SS1P.

Reactions of this sort will often result in the formation of conjugates containing one or more polymers attached to the SS1P. As will be appreciated, it will often be desirable to isolate the various fractions and to provide a more homogenous product. In most aspects of the invention, the reaction mixture is collected, loaded onto a suitable column resin and the desired fractions are sequentially eluted off with increasing levels of buffer. Fractions are analyzed by suitable analytical tools to determine the purity of the conjugated protein before being processed further. Regardless of the synthesis route and activated polymer selected, the conjugates will conform to Formula (I) as defined herein. Some of the preferred compounds which result from the synthetic techniques described herein include:

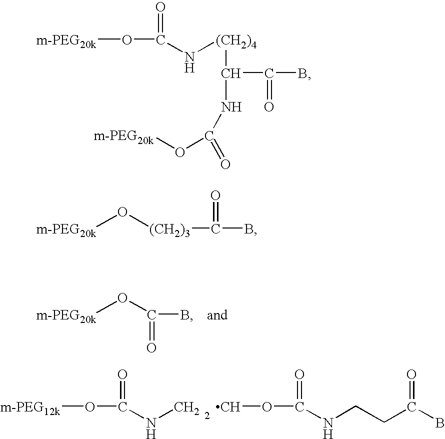

wherein B is SS1P.

Still further conjugates made in accordance with the present invention include:

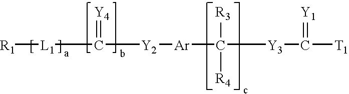

wherein all variables are the same as that set forth above and T₁ is one of

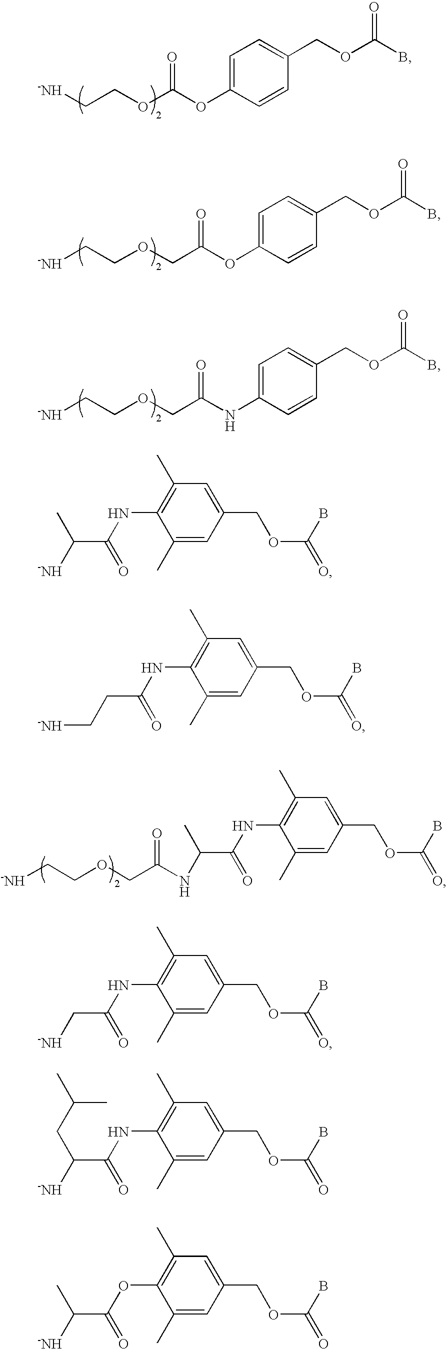

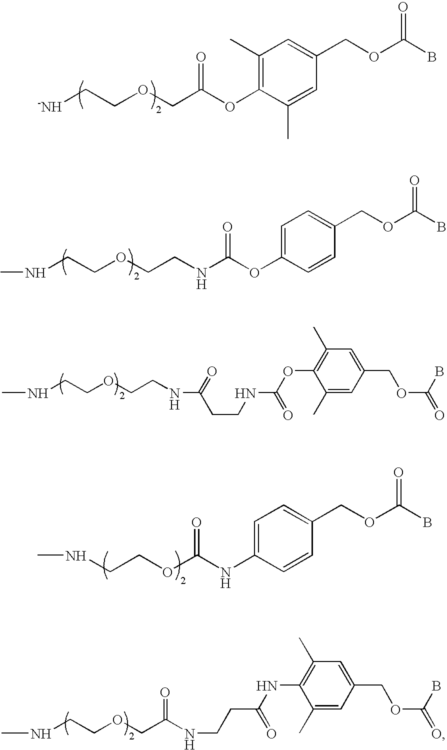

wherein B is SS1P.

Further conjugates include:

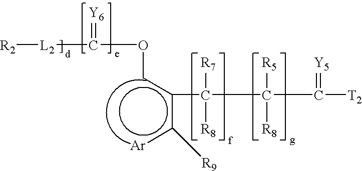

wherein T₂ is

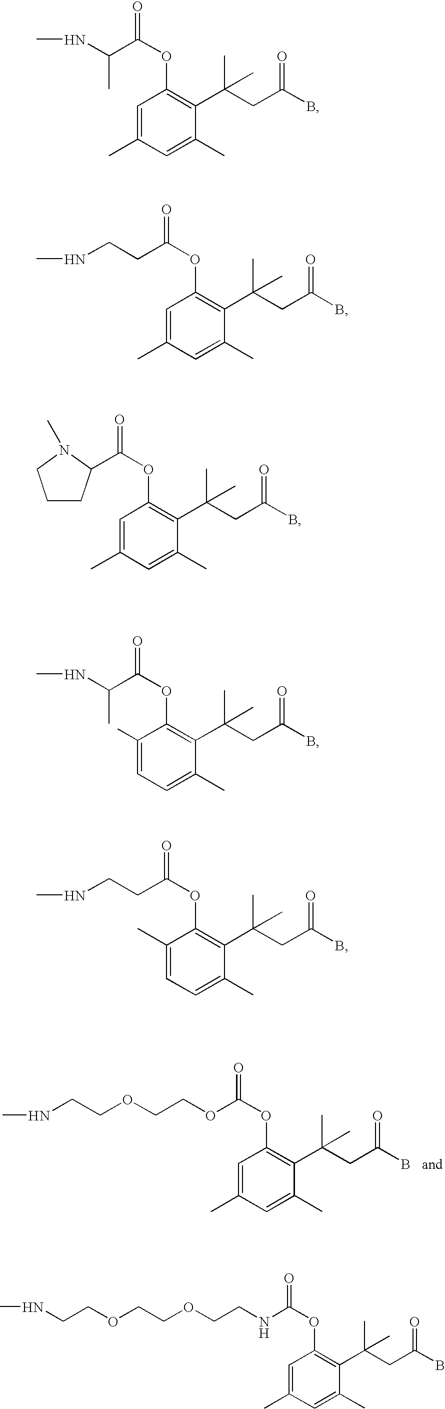

wherein B is SS1P.

A particularly preferred conjugate is:

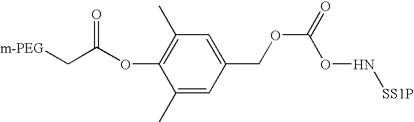

wherein the molecular weight of the mPEG is from about 10,000 to about 40,000.

When the bicine-based polymer systems are used, two preferred conjugates are:

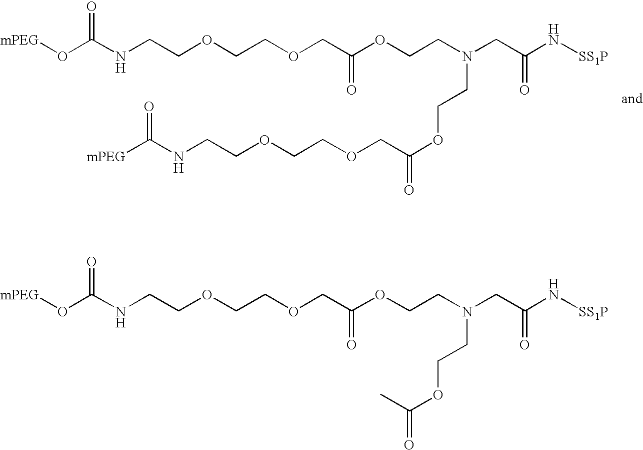

wherein the molecular weights of the mPEG are the same as above.

### Pharmaceutical Compositions and Administering Immunotoxin

The pharmaceutical compositions of the invention may include a “therapeutically effective amount” or a “prophylactically effective amount” of the polymer-linked immunotoxin. A “therapeutically effective amount” refers to an amount effective, at dosages and for periods of time necessary, to achieve the desired therapeutic result. A therapeutically effective amount of the polymer-linked immunotoxin may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Thus, a typical dosage range for the polymer-linked immunotoxin, e.g., a PEG-SS1P conjugation for intravenous administration would a quantity that would deliver the equivalent of from about 0.1 to 10 mg of PEG-free SS1P per patient per day. Dosages that would deliver the equivalent of from 0.1 up to about 100 mg of PEG-free SS1P per patient per day may be administered.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of PEG-SS1P of the invention is from about 0.1 to about 20 mg/kg, more preferably, from about 1 to about 10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In a further preferred embodiment, the polymer-linked immunotoxins of the invention are employed for treating and/or diagnosing tumors or cancers to which the anti-mesothelin immunotoxin will bind. Thus, polymer-linked immunotoxin is administered by art-known methods, to an animal or person having a tumor or cancer responsive to treatment by the polymer-linked immunotoxin.

Actual methods for preparing administrable compositions will be known or apparent to those skilled in the art and are described in more detail in such publications as REMINGTON'S PHARCMACEUTCAL SCIENCE, 19TH ED., Mack Publishing Company, Easton, Pa. (1995). For example, the polymer-linked immunotoxins of the invention can be incorporated into pharmaceutical compositions suitable for administration to a subject, e.g., an animal or person in need of such administration. Typically, the pharmaceutical composition comprises a polymer-linked immunotoxin having at least one type of binding specificity, and a pharmaceutically acceptable carrier.

The term, “pharmaceutically acceptable carrier” includes any and all solvents, dispersion media, coatings, antimicrobial, e.g., antibacterial and antifungal agents, isotonic and absorption delaying agents, and the like, that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition.

Pharmaceutically acceptable carriers may further comprise minor amounts of auxiliary substances such as wetting or emulsifying agents, preservatives or buffers, which enhance the shelf life or effectiveness of the antibody or antibody portion.

The inventive polymer-linked immunotoxins are optionally prepared in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular).

In a preferred embodiment, the polymer-linked immunotoxin is administered by intravenous infusion or injection. In another preferred embodiment, the antibody is administered by intramuscular or subcutaneous injection. Administration via inhalation, as a spray, aerosol or mist is also contemplated where that route is advantageous, e.g., for systemic absorption and/or local action within the respiratory system.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization.

Generally, dispersions are prepared by incorporating the active polymer-linked immunotoxin into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The polymer-linked immunotoxins of the present invention can be administered by a variety of methods known in the art, although for many therapeutic applications, the preferred route/mode of administration is intravenous injection or infusion. As will be appreciated by the skilled artisan, the route and/or mode of administration will vary depending upon the desired results.

In certain additional preferred embodiments, if the tumor or cancer to be treated is present in the lining of the mouth, esophagus or other parts of the gastrointestinal system, the polymer-linked immunotoxin can be orally administered in a suitable pharmaceutical composition for treating such gastrointestinal tumors or cancers, for example, admixed with an inert diluent or an assimilable edible carrier. The polymer-linked immunotoxin (and other optional ingredients, if desired) are optionally enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer the polymer-linked immunotoxins of the invention by other than parenteral administration, it may be necessary to coat the compound with, or co-administer the compound with, a material to prevent its inactivation.

Supplementary anti-tumor agents can also be incorporated into the pharmaceutical compositions. In certain embodiments, a polymer-linked immunotoxin of the invention is co-formulated with and/or co-administered (simultaneously or sequentially), in combination with one or more additional therapeutic modalities (e.g., chemotherapeutic agents, radiation, surgery and combinations thereof) that will provide additive, synergistic or supplementary therapeutic or diagnostic activity for a tumor or cancer. Such additional modalities optionally include radiation, such as X-ray, gamma ray, or particle beam radiation, laser light and/or infrared radiation. Anti-tumor agents that can be combined with the polymer-linked immunotoxin of the invention include, simply by way of example, Taxol™, cyclophosphamide, melphalan, levamisol NAC, 5 fluorouracil, methotrexate, cisplatin, carboplatin, cyclophosphamide and ifosfamide, bleomycin, mamsa, streptozotocin, hydroxyurea, etoposide, deoxycoformycin, fludarabine, chlorodeoxyadenosine, doxorubicin and daunorubicin, paclitaxel, vincristein, vinblastine, mAMSA, ThioTEPA, epirubicin, 5-fluorouracil, 6-mercaptopurine, L-phenylalanine mustard, MDR, MRP, topoisomerase I, topoisomerase II, toxal, vincristine, vinblastine, vindesine, VP-16, VM-26, dactinomycin, doxorubicin, idarubicin, mithramycin, mitomycin-c, bleomycin, methotrexate, with leucovorin, methotrexate, 5-fluorouracil, 5-fluorouracil w/leucovorin, 5-fluorodeoxyuridine, 6-mercaptopurine, 6-thioguanine, cytarabine, 5-azacytidine, hydroxyurea, deoxycoformycin, fludarabine, cyclophosphamide, ifosfamide and mesna, melphalan, CCNU, MeCCNU, BCNU, chlorambucil, CBDCA (carboplatin), aziridinylbenzoquinone (AZQ), DTIC (dacarbazine), mAMSA, procarbazine, hexamethylmelamine, and mitoxantrone, to name but a few such agents.

### Indications for the PEG-SS1P Immunotoxin

The inventive polymer-linked SS1P immunotoxin is targeted to cells or tissue expressing mesothelin protein. Preferably, the polymer is a polyethylene glycol, as discussed supra. Since mesothelin is expressed primarily by tumor or cancer cells, PEG-SS1P is primarily an anti-cancer agent and is contemplated to be used in treating animals that will benefit from such anti-cancer treatment. Animals that will benefit are any animals that have a tumor or cancer that expresses mesothelin protein.

In addition to humans, animals to be treated include vertebrates, such as mammals, avians, and fish. Among the vertebrates, companion animals, including cats, dogs, pet birds and the like, are also contemplated to benefit from administration of PEG-SS1P for treating tumors or cancers expressing mesothelin.

Methothelin is found in normal (non-tumor) mesothelial cells lining body cavities, but is not present in important organs, such as the: heart, lungs, liver, kidneys and nervous tissue. Tumors that are known to express mesothelin antigen include, mesotheliomas, ovarian cancers and some squamous cell carcinomas. Mesothelin is present in more than 90% of human epithelial mesothliomas and more than 90% of human pancreatic adenocarcinoma and in from 66-74% of human non-mucinous ovarian cancers.

It is contemplated that tumors will be tested for mesothelin antigen expression when initially diagnosed and/or during the course of treatment. Reagents and assays for detecting mesothelin (a/k/a mesothelium antigen) are described, for example, by U.S. Pat. No. 6,083,502, incorporated by reference herein.

Thus, the indications for administering the inventive PEG-SS1P to a patient (human or nonhuman) includes a diagnosis of a mesothelioma, ovarian cancer and squamous cell carcinoma, as well as any tumor that is confirmed to express mesothelin, or that is confirmed to express any antigen that binds to an anti-mesothelin antibody.

### EXAMPLES

The following examples serve to provide further appreciation of the invention but are not meant in any way to restrict the effective scope of the invention.

### Example 1

### Preparation of Recombinant Immunotoxin

### The Expression Vectors

The SS1P immunotoxin was constructed as a disulfide-linked (“ds”) dimer. Each of the two components was separately expressed, isolated and renatured under conditions promoting ds dimer formation.

BL21(DE3)/pPSC7-7 cm Cell Line

The anti-mesothelin heavy chain variable domain (“SS1-PE38V_{H}”) was expressed in culture by BL21(DE3) host cells containing a pPDC7-4 cm plasmid (FIG. 1; SEQ ID NO:1). This is the BL21(DE3)/pPSC7-7 cm cell line. The DNA molecule encoding the SS1-PE38V_{H} polypeptide is according to SEQ ID NO: 6, and the SS1-PE38V_{H} polypeptide sequence is as follows.

| (SEQ ID NO: 5) | |
|---|---|
| MQVQLQQSGPELEKPGASVKISCKASGYSFTGYTMNWVKQSHGKCLEWIGLITPYNGASS | 60 |
| YNQKFRGKATLTVDKSSSTAYMDLLSLTSEDSAVYFCARGGYDGRGFDYWGQGTTVTVSS | 120 |
| kasggpeggslaaltahqachlpletftrhrqprgweqleqcgypvqrlvalylaarlsw | 180 |
| nqvdqvirnalaspgsggdlgeaireqpeqarlaltlaaaeserfvrqgtgndeagaang | 240 |
| padsgdallernyptgaeflgdggdvsfstrgtqnwtverllqahrqleergyvfvgyhg | 300 |
| tfleaaqsivfggvrarsqdldaiwrgfyiagdpalaygyaqdqepdargrirngallrv | 360 |
| yvprsslpgfyrtsltlaapeaageverlighplplrldaitgpeeeggrletilgwpla | 420 |
| ertvvipsaiptdprnvggdldpssipdkeqaisalpdyasqpgkppredlkz | 473 |

Residues representing the V_{H} domain are in UPPER case, the PE38 domains are in lower case. A serine at position 45 of SEQ ID NO:5 has been changed to cysteine (underlined) to provide a site for the formation of a disulfide bond to link with the separately produced V_{L}. The artisan will note that the replaced serine is actually designated as position 44 of the V_{H} domain, based on Kabat et al., 1987 “Sequences of proteins of immunological interest,” 4th ed., U.S. Dept. Health and Human Services, Public Health Services, Bethesda, Md., incorporated by reference herein).

The V_{L} polypeptide is encoded by a DNA molecule having a sequence according to SEQ ID NO: 8, and the polypeptide has the following sequence.

| (SEQ ID NO: 7) | |
|---|---|
| MDIELTQSPAIMSASPGEKVTMTCSASSSVSYMHWYQQKSGTSPKRWIYDTSKLASGVPG | 60 |
| RFSGSGSGNSYSLTISSVEAEDDATYYCQQWSKHPLTFGCGTKLEIKZ. | 108 |

### A glycine at position 100 (Kabat Id.) has been changed to cysteine (underlined) to form a disulfide bond with the V_{H} domains of the SS1-PE38V_{H} polypeptide.

### Example 2A

### Culture of the E-Coli Expression Vectors

The BL21(DE3) E. coli strain carrying the plasmid expression vector containing cDNA of SS1-PE38V_{H} was grown at 37° C., in Superbroth medium supplemented with kanamycin (10 μg/ml) and chloramphenicol (25 μg/ml) in batch culture in a 5L fermenter with 4.5 L of medium for 5-6 hours until the density reached an OD₆₀₀ value of 5-8. Cells were then induced with 5 mM IPTG for 1.5 hours. Grown cells containing the expressed SS1(dsFv)-PE38 immunotoxin and SS1V_{L} were harvested by centrifugation in a Beckman centrifuge (model Avanti J-20I, Fullerton, Calif.) using a JLA8.1000 rotor for 20 minutes at 4° C. at 7000 rpm. A typical yield was from 15-20 g (wet weight) of cells per liter of culture fluid.

The BL21(DE3) E. coli strain carrying expression vector containing cDNA of SS1-V_{L} was grown a 7° C., in Superbroth medium supplemented with kanamycin (10 μg/ml) and chloramphenicol (25 μg/ml) in batch culture in a 5L fermenter_with 4.5 L of medium for 15-16 hours until the density reached an OD₆₀₀ value of 10-15. Cells were then induced with 5 mM IPTG for 1.5 hours. Grown cells containing the expressed SS1V_{L} were harvested by centrifugation in a Beckman centrifuge (model Avanti J-20I, Fullerton, Calif.) using a JLA8.1000 rotor for 20 minutes at 4° C. at 7000 rpm. A typical yield was 24-28 g (wet weight) of cells per liter of culture medium.

### Example 2B

### Purification of SS1P Proteins from Cultured E. Coli

SS1(dsFv)-PE38 was expressed in E. Coli host cells cultured as described above and purified by the following method.

### 1. Reagents and Buffers

The following reagents were employed in the purification of the SS1P immunotoxin from cultured host cells.

Lysozyme; dithioerythritol (DTE); glutathione, oxidized form (GSSG); L-arginine-HCl; Triton X-100; urea; 0.1 N, 1N NaOH.

Sterile solution: 1 M Tris-HCl, pH 7, 4; 1 M Tris-HCl, pH 8.0; 0.5 M EDTA, pH 8.0; 5 M NaCl; PBS (1X) without calcium and magnesium.

The following buffers were employed in the purification of the SS1P immunotoxin from cultured host cells.

| TES Buffer | TE 50/20 Buffer |
|---|---|
| 50 mM Tris-HCl, | 50 mM Tris-HCl, |
| pH 7.4 | pH 7.4 |
| 20 mM EDTA | 20 mM EDTA |
| 100 mM NaCl | |
| Solubilization buffer | Refolding buffer |
| 6 M Guanidine-HCl | 0.1 M Tris |
| 0.1 M Tris-HCL, pH 8.0 | 0.5 M L-arginine-HCl (105 g/L) |
| 2 mM EDTA | 2 mM EDTA |

The refolding buffer was prepared as follows. The pH was adjusted to 10.5 with 10 N NaOH, the temperature was equilibrated to 4° C., and 0.9 mM GSSG (Glutathione, oxidized form) (551 mg/L) was added before the refolding.

| Dialysis buffer | Buffer A |
|---|---|
| 20 mM Tris-HCl | 20 mM Tris-HCl, |
| pH 7.4 | pH 7.4 |
| 100 mM urea* | 1 mM EDTA |

*the urea was not added into the buffer more than 12 hrs before use.

### 2. Preparation of Inclusion Bodies

1. 10-12 grams (wet weight) of the bacteria pellets containing SS1V_{L} or SSV_{H}-PE38 in 180 ml TES per 250 ml centrifuge bottle (for the Sorval GSA rotor) was resuspended.

2. A tissuemizer (tissue homogenizer) was used in order to thoroughly resuspend the pellets and avoid lump formation.

3. 50 ml of a lysozyme stock solution (8 mg/ml) was prepared in TES buffer.

4. 8 ml of the lysozyme stock solution was added to each centrifuge bottle and was immediately shaken well.

5. The bottles were incubated for 60 min at room temperature with intermittent shaking.

6. The settled pellet material was resuspended using a tissuemizer.

7. 20 ml of 25% Triton X-100 (the stock solution was prepared a day ahead, since the two liquids mix slowly) was added to each centrifuge bottle.

8. The samples were incubated for 30 min at room temp with frequent and thorough shaking.

9. A tissuemizer was used to break up DNA mats, if present and then, the samples were centrifuged at 13000 rpm for 50 min at 4° C.

10. The pellets were then completely resuspended in TE 50/20 buffer, to which 20 ml of 25% Triton X-100 was added.

11. The samples were then centrifuged at 13000 rpm for 50 min at 4° C.

12. Steps 10-11 were repeated three times.

13. The pellets were then resuspended in TE 50/20 buffer.

14. The samples were then centrifuged at 13000 rpm for 50 min at 4° C.

15. Steps 13-14 were repeated three times.

### 3. Solubilization of Inclusion Bodies

1. The wet inclusion body (“IB”) pellets (about 100 mg protein/gram of IB) obtained as described above were dissolved in 5-10 ml of solubilization buffer.

2. A tissuemizer was used to resuspend the inclusion bodies.

3. The suspended IB samples were then incubated overnight at room temp.

4. The protein concentration was determined using the Pierce Coomassie Blue Plus assay (modified Bradford). The same final concentration of guanidine was used for the reference standard protein (BSA), and for the IB protein. The inclusion body solution was then 20-fold diluted with the solubilization solution, then the protein concentration assay was conducted.

5. The inclusion bodies were stored at −80° C., if not used right away.

### 4. Renaturation in DTE/GSSG

a. Dry dithioerythritol (“DTE”) was weighed out and added to inclusion body solution obtained from step 4 of section 3, above, to a final concentration of 65 mM (10 mg/ml).

b. The solution was incubated for 2 hrs at room temp.

c. The protein concentration of the DTE-reduced inclusion bodies was then re-measured. Typically, the inclusion bodies were more completely solubilized after DTE-reduction, providing an increase in the measured protein concentration.

d. The inclusion body solution was diluted to 10 mg/ml with the solubilization solution containing 10 mg/ml of DTE.

e. The inclusion bodies of V_{L} and V_{H}-PE38 were then mixed together in a weight ratio of 1:2.

f. The inclusion body mixture was then 100-fold diluted with the refolding buffer.

g. The refolding solution was then incubated at 4° C. for 36-42 hours without stirring.

### 5. Concentration and Dialysis

a. The pH of the refolding solution was adjusted down to 8.5. Then the refolding solution was 10-fold concentrated using Areicon H1P39043 hollow-fiber cartridges that were cleaned with 0.1 N NaOH, then washed with plenty of Milli Q water to neutral pH.

b. The refolding solution was dialyzed against 20 mM Tris-HCl, pH 7.4 containing 0.1 M urea using the same cartridges. The dialysis was concluded when the conductivity was reduced to 10 mMho, or lower.

c. The concentrated solution was then centrifuged for 30 min. at 13,000 rpm. The supernatant was filtered through a 0.2 μm membrane filter.

d. The conductivity was then adjusted down to 7 mMho or lower with Buffer A.

### 6. Purification

a. A Q-Sepharose column (8-10 ml per liter refolding solution) was prepared. The column was washed with 1 N NaOH followed with plenty of water (at least 10 column volumes), and then equilibrated with buffer A. The filtrated supernatant was then loaded onto the column. The column was washed with 5-bed volume of 0.1 M NaCl in buffer A. The column was then eluted with 4-bed volume of 0.3 M NaCl in buffer A.

b. The fractions were analyzed by SDS-PAGE and pooled in the first major peak.

c. A Mono-Q column was treated the same way as was for the Q-Sepharose column. The pooled Q-Sepharose fractions were 5-fold diluted with buffer A and loaded on the column. The protein was then eluted with a NaCl gradient (0.1-0.3 M) in buffer A (20 bed volume).

Most of the fractions contain SS1(dsFv)-PE38, but only the fractions of the major peak have high activity, The major peak is separated by a narrow cliff. One fraction was collected before the cliff and most of the fractions after the cliff, except the edge of the major peak. (fraction size is one half of the bed volume)

d. The pooled Mono Q fractions were 5-fold diluted and reloaded onto a smaller Mono Q column, then eluted with 0.5 M NaCl in buffer A.

e. All of the fractions containing protein were pooled and loaded onto a Superalex 200 column previously washed with 0.1 N NaOH and equilibrated with PBS. The column was eluted with PBS. Major peak fractions were pooled and protein concentration determined. The concentration was then adjusted to 1 mg/ml. Aliquots were prepared and frozen at −80° C.

The final yield was around 12% (based on the amount of the inclusion body proteins in the refolding).

### 7. Cytotoxicity Assay

Activity of the above-obtained SS1P protein was confirmed by a cytotoxicity assay using 15,000 cells/well. A431K5 cells were seeded in a 96-well plate. Samples of either native SS1P or of PEG-conjugates of SS1P were added to the cells the next day at different concentrations. Tritiated leucine was added 20 hours after addition of the SS1P/PEG-conjugates and incubated at 37° C. for 2 hours. The IC₅₀ value was around 0.8 ng/ml.

### 8. Endotoxin Assay

The above obtained SS1p protein was tested for endotoxin, and the results were less than 3 EU/mg protein using the LAL kits sold by Associates of Cape Cod, Inc. for assay.

### Example 2C

### Alternative Purification of SS1P Immunotoxin from Cultured E. Coli

An alternative method of isolating the SS1P proteins from cultured E. coli host cells was also developed. This alternative method provides improved yields.

### 1. Processing of Host Cells

Host Cells expressing SS1-PE38V_{H} and SS1V_{L} were harvested as follows. Cells were harvested at 12,000×g (7000 rpm in Beckman) for 20 minutes at 4 degrees C. Wet weights were about 42.5 g for SS1-PE38V_{H} and 22.5 g for SS1V_{L}, respectively. Cells were stored at −20 degrees C.

### 2. Preparation of Inclusion Bodies (“IB”) from SS1V_{L}-Expressing Cells

IB was isolated from cells expressing SS1V_{L} as follows.

The harvested cells were resuspended with Resuspension Buffer (“RB”) (TES: Tris, EDTA, Sodium Chloride, pH 7.4, as defined supra) in a volume of 20 ml/g of cells.

The resuspended cell mass was homogenized at 6000 rpm for 5 min, with a homogenizer, and then screened by passing the cell suspension through a metal mesh of 250 micrometer opening USA Standard Test Sieve, #60. The screened cells were then disrupted by Microfluidization, for 3 cycles, on ice, followed by centrifugation at 12,000×g (7000 rpm in Beckman) for 20 minutes at 4 degrees C.

1/10th volume of 25% Triton X-100 (in water) was added to the pellets, following by incubation, with stirring, for 30 min at room temperature (“RT”), followed by centrifugation of the incubated material at 12,000×g (700 rpm in Beckman) for 20 minutes at 4 degrees C. The resulting pellets were washed with TES, 20 ml/g and then homogenized.

The above washing step was then repeated, two more times, but the incubation with stirring was reduced to 5 minutes.

The resulting pellets were then washed without Triton X-100, by adding TES, 20 ml/g and homogenizing, then incubating, with stirring, for 5 min at RT. Incubation was followed by centrifugation at 12,000×g (7000 rpm in Beckman) for 20 minutes at 4 degrees C.

The above washing step (without Triton X-100) was then repeated two more times.

The weight of the resulting pellets was 1.85 g

The resulting SS1V_{L} IB material was storage at −20/−80 degrees

### 3. Preparation of IB from SS1-PE38V_{H}-Expressing Cells

The harvested cells expressing SS1-PE38V_{H} were resuspended with RB: TES, Tris, EDTA, Sodium Chloride, pH 7.4, at a ratio of 20 ml/g of cells. The resuspended cell mass was homogenized at 6000 rpm for 5 min, with a homogenizer, and then screened by passing the cell suspension through a metal mesh of 250 micrometer opening, USA Standard Test Sieve, #60. The screened cells were then disrupted by Microfluidization, for 3 cycles, on ice, followed by centrifugation at 12,000×g (7000 rpm in Beckman) for 20 minutes at 4 degrees C., and then treated with Triton X-100 and washed by repeated centrifugation, using the protocol provided above for the preparation of the SS1V_{L}.

Wet weight of the final pellet 5.85 g.

The collected IB material was stored at −20/−80 degrees C.

### 4. Solubilization of the SS1V_{L} IB Material

In order to convert the collected SS1V_{L} IB material into a soluble SS1V_{L} protein, the collected IB material was subjected to the following process.

Weight of IB was 1.85 g

Solubilization Buffer (6M GuHCl in 100 mM Tris.HCl, pH 8.0, 1.0 mM EDTA (GTE Buffer)

Buffer Volume was 5-10 ml for 1 g of IB material.

The collected IB material was extracted, with stirring, overnight, at 25 degrees C. Once the material was dissolved in GuHCl, the appearance was straw colored, with little turbidity. The GuHCl solution was then centrifuged at 14,000× rpm in a Sorvall for 45 minutes, at 15-20 degrees C., and the supernatant was collected.

Volume 15.0 ml

Protein Concentration 15.0 mg/ml

Adjust Protein Concentration to 10 mg/ml with GTE buffer

### 5. Solubilization of SS1-PE38V_{H} IB

The same reagents and process as described above for the SS1V_{L} IB material was applied to the collected SS1-PE38V_{H} IB material.

Weight of IB 5.85 g

Volume 22.0 ml

Protein Concentration 20.0 mg/ml

Adjust Protein concentration 10 mg/ml with GTE buffer

### 6. Denaturation of V_{L}

22.0 ml of the solubilized SS1V_{L} IB material, prepared as described above, was mixed with 10 mg/ml of DTE and incubated, overnight, at RT without stirring.

### 7. Denaturation of SS1-PE38V_{H}

44.0 ml of the solubilized SS1-PE38V_{H} IB material, prepared as described above, was mixed with 10 mg/ml of DTE and incubated, overnight, at RT without stirring.

### 8. Refolding to Form Disulfide-Linked Complete SS1P [dsFvSS1P]

In order to form the final SS1P protein (dsFvSS1p or SS1P), the denatured SS1V_{L} and SS1-PE38V_{H} were mixed under conditions promoting refolding and formation of a disulfide linkage between the engineered cystine residues present on both the SS1V_{L} and SS1-PE38V_{H} moieties.

The Refolding Buffer was Tris HCl 100 mM, pH 10.0, 25 mM EDTA, NaCl 0.1 M, GSSG 551 mg/L, 0.5 M Arginine

22.0 ml of the denatured SS1-V_{L} solution was admixed with 44.0 ml of denatured SS1-PE38V_{H} in a molar ratio of 1:2 (SS1-V_{L}:SS1-PE38V_{H}) for a total volume of 66.0 ml. The admixture was stirred gently but thoroughly for less than 5 minutes.

100 volume of Refolding Buffer was then added for each 1 volume of the admixed denatured IB, followed by rapid stirring for 2 minutes at RT. The SS1-V_{L} and SS1-PE38V_{H} proteins were allowed to refold for 45 h without stirring at 4° C.

Total Volume of Refolding mixture was 6600.0 ml.

Total protein Conc. (mg/ml) 0.1 mg/ml

Total Protein 660 mg

Purity of SS1-PE38V_{H} 58%

Purity of SS1-V_{L} 44.5%

Estimated pure SS1-PE38V_{H} 255.2 mg

Estimated pure SS1-V_{L} 97.9 mg

Estimated Yield of SS1P formation 22-25%

### 9. Adjustment of pH of Renatured SS1P

Total volume of buffer with renatured SS1P was 6600.0 ml.

Conc. of HCl was 12N.

Initial pH of buffer with renatured SS1P was 10.5.

Volume of NaOH added was about 300 ml.

Final pH of buffer with renatured SS1P was 8.5.

Final Volume of the sample was 6900.0 ml.

### 10. Clarification of Renatured SS1P

Filter Unit was ACROPAK 1000 Capsule (0.8u/0.45u), PALL

Initial Volume was 6900.0 ml.

Final Volume was 6900.0 ml.

Protein Concentration was 0.095 mg/ml.

Total protein was 6600.0 mg.

Purity (based on SDS-PAGE) 20-25%

Step yield>95%

Protein recovery>95%

### 11. MEP-HyperCel Chromatography

The MEP-HyperCel resin specifically binds to the SS1P-V_{L} domain. The renatured SS1P was purified using MEP-HyperCel column chromatography without any further processing of the refolded protein. The refolded SS1P was allowed to bind to the MEP-HyperCel column by passing the refolding solution. Proteins bound non-specifically were then removed by low pH (pH 5.0) wash. The fully renatured SS1P was eluted from the column with pH 4.0 buffer in the presence of 250 mM NaCl. The pH of the eluted SS1P sample was changed to 7.4 using 0.1N NaOH and the high salt was removed by dialysis against 10 volume of 20 mM phosphate buffer at pH 7.4. SS1P was further purified using a SourceQ-30 column. The column was pre-equilibrated with 20 mM phosphate at pH 7.4 containing 25 mM NaCl. Once the sample was loaded, non-specific proteins were washed off and SS1P was eluted with a linear salt gradient. The fractions were analyzed by SDS-PAGE and fractions with highest purity were pooled. For PEGylation, purified SS1P was dialyzed against the desired buffer.

The Resin was MEP-HyperCel (Ciphergen, Inc.).

Reported Binding Capacity is 10 mg/ml to 20 mg/ml.

Bed Volume 100 ml.

Flow Rate 5-10 ml/min.

Equilibration Buffer was 20 mM Tris, pH 7.6, NaCl 250 mM and 1 mM EDTA.

Equilibration Volume>5×CV [CV is column volume].

Protein Load was 353 mg.

Wash 500 ml (5×CV).

Wash Buffer was 20 mM Tris, pH 7.6, NaCl 250 mM and 1 mM EDTA.

### 12. Low pH Wash

Buffer was Na-acetate, 50 mM, pH 5.0, and 250 mM NaCl.

Buffer Volume was 500 ml (5×CV).

Elution was with Na-acetate, 50 mM, pH 4.0, and 250 mM NaCl.

Elution Volume was 10×CV.

Volume Collected was 660 ml.

Protein Concentration was 0.098 mg/ml.

Total Protein was 64.7 mg.

Purity (based on SDS-PAGE) was 85%.

Yield was 18.3% (based on total protein).

### 13. Adjustment of pH with NaOH

Initial pH of the solution: 4.0.

Concentration of NaOH was 1.0 N.

Volume of NaOH added was 31 ml

Final pH was 7.4.

Final volume was 691 ml.

### 14. Clarification (When Necessary)

Centrifugation at 13,000 rpm for 30 minutes

Supernatant was about 690 ml.

Protein Concentration 0.093 mg/ml

Total Protein 64.17 mg

Protein Recovery 99.1%

Step Yield 99%

### 15. Dialysis

The MEP-HyperCel-eluted sample was dialyzed against 10-volume of 20 mM phosphate buffer at pH 7.4 overnight at 4° C. to remove excess salt from the sample.

Initial Volume 691 ml.

Final Volume 726 ml.

Initial Conductivity 35-40 mS/cm.

Final Conductivity 6-8 mS/cm.

Protein Concentration 0.09 mg/ml.

Total Protein 64 mg.

Protein Recovery 99%.

### 16. Source Q-30 Chromatography

Resin was Source Q-30 [GE HealthCare/formerly Amersham/Pharmacia].

Binding Capacity 15 mg/ml to 25 mg/ml.

Bed Volume 8 ml.

Flow Rate 4 ml/min.

Equilibration Buffer was Tris.HCl, 20 mM, pH 7.4, 1 mM EDTA, 20 mM NaCl.

Equilibration Volume 5 C×V.

Protein Load 45 mg.

Wash was with 5 C×V.

Wash Buffer (Buffer A: 20 mM NaPhosphate, pH 7.4 and 50 mM NaCl).

Elution Buffer (Buffer B: 20 mM NaPhosphate, pH 7.4 and 500 mM NaCl).

Elution was by Gradient Elution, between 50 mM NaCl and 225 mM NaCl.

Elution Volume 20 C×V.

Volume Collected 38 ml.

Protein Concentration 0.75 mg/ml.

Total Protein 28.5 mg.

Purity (based on (sp. Act) gel scanning>95%.

Step Yield was 73.8%.

Final Yield was 8.1%.

### Adjustment of Final Purification Product

The purified protein was dialyzed against buffers suitable for a specific PEGylation process. For instance, SS1P intended for DGA-2 or BCN3—PEGylation, was dialyzed against phosphate buffer containing 50 mM sodium phosphate, pH 7.8 and 50 mM NaCl or, only phosphate buffer containing 0.1 M sodium phosphate at pH 7.6.

### Example 3

### Preparing Releasable PEG-SS1P Conjugates

In PEGylation reactions employing releasable linkers such as DGA2, RNL-8a, and BCN, SS1P at 1.5-2.5 mg/mL was PEGylated in 0.05-0.1 M sodium phosphate, pH 7.6, 25° C. With fast stirring without creating foam, the activated PEG powder at 10:1 to 50:1 reaction molar ratio of PEG to SS1P was added to SS1P solution at 1 g/min rate. The reaction was continued at 25° C. for 1 hour and quenched by adding glycine. Immediately after quenching, the solution pH was lowered to 6.5 with sodium phosphate, mono basic, and the conjugate was purified on size exclusion column (Superdex 200 or 75 Hiload, Amersham, N.J.) equilibrated in 20 mM sodium phosphate, pH 6.5, 140 mM NaCl.

Alternatively, the conjugate was purified on an anion exchange column (Q Sepharose Fast Flow column, Amersham, N.J.) using 10 mM sodium phosphate, pH 7.4 as an equilibrium buffer and 0.3 M sodium chloride in 10 mM sodium phosphate, pH 7.4 as a gradient elution buffer or 10 mM Tris, pH 7.6 as an equilibrium buffer and 0.3 M sodium chloride in 10 mM Tris, pH 7.6 as a gradient elution buffer. The column fractions containing the peak were combined and dialyzed against PBS, pH 6.5. The sample was concentrated to about 1 mg/mL using Centriplus 30k (Millipore, Mass.) and passed through a sterile filter (Acrodisc Syringe Filter, 0.2 μm HT Tuffyn membrane, Pall, Mich.).

The protein concentration was determined by bicinchoninic acid assay (PIERCE, IL) using bovine serum albumin as a standard and the cytotoxicity was analyzed on A431/K5 cells. The number of PEG polymers per SS1P molecule was estimated by SDS-PAGE (precast 4-20% SDS non-reducing gel, Invitrogen, Calf.). The release of SS1P from releasable PEG-SS1P was conducted by incubation at pH 8.5 buffer, 37° C. for 4 hours. The purity of the product was analyzed on a TSK gel filtration column equilibrated in 50 mM sodium phosphate, pH6.5, 150 mM NaCl (G4000SWXXL), 7.8×30 cm, 8 μm, Tosoh Biosciences). The peak area was calculated at 220 nm.

### Example 3A

### Preparing Releasable DGA2-SS1P

To 4-mL of 2.5 mg/mL SS1P in 0.1 M sodium phosphate, pH 7.6 solution was added 48 mg (25:1) DGA2-12k-NHS powder (Enzon, EZ1064/E1029-154A). The reaction was continued at 25° C. for 60 min, quenched with glycine at 10:1 molar ratio of glycine to PEG. The conjugate was purified on Superdex 200 Hiload column equilibrated with 20 mM sodium phosphate, pH 6.5, 140 mM NaCl. The fractions of the peak were combined and filtered through a 0.2 μm sterile filter.

TABLE 1
Analysis of Releasable DGA2-SS1P

| Overall | | | |
|---|---|---|---|
| yield (%) | Purity (% major peak area) | Composition | IC₅₀ (ng/mL) |
| 100 | 99 | 4-6 | 2.73 |
| | | PEG/SS1P | |

### Example 3B

### Preparing of Releasable 24K BCN3—SS1P

To 2.2-mL of 1.8 mg/mL SS1P in 0.1 M sodium phosphate, pH 7.6 solution was added 38-mg (25:1) BCN3-24k-NHS powder (Enzon, 1165-170). The reaction was continued at 25° C. for 60 min and quenched with glycine at 10:1 molar ratio of glycine to PEG. The conjugate was purified on Superdex 200 Hiload column equilibrated in 20 mM sodium phosphate, pH 6.5, 140 mM NaCl. The fractions of the peak were combined and filtered through a 0.2 μm sterile filter. There were 2-4 PEG per SS1P molecule.

TABLE 2
Analysis of releasable 24K BCN3-SS1P

| | Purity | | | IC₅₀ |
|---|---|---|---|---|
| Overall | (% major | | IC₅₀ | after 4 hrs at pH 8.5, |
| yield (%) | peak area) | Composition | (ng/mL) | 37° C. (ng/mL) |
| 33 | >90 | 2-4 | 1.09 | 0.56 |
| | | PEG/SS1P | | |

### Example 3C

### Preparing Releasable 24K BCN3—SS1P

To 5.9 mL of 1.7 mg/mL SS1P in 0.1 M sodium phosphate (pH 7.6) solution was added 152 mg BCN3-24k-NHS powder (40:1 reaction molar ratio) (Enzon, 1165-170). After 60-min reaction at 25°, the reaction mixture was diluted 15-fold with H₂O to 1 mS conductivity and purified on a Q column (4-mL bed volume, 1×5.3 cm). The equilibration buffer was 10 mM sodium phosphate, pH 7.6 and the gradient elution buffer was composed of 10 mM sodium phosphate, pH 7.6, 0.3 M NaCl. The fractions were combined and passed through a 0.2 μm sterile a filter.

TABLE 3
Analysis of releasable BCN3-SS1P

| Overall | | | |
|---|---|---|---|
| yield (%) | Purity (% major peak area) | Composition | IC₅₀ (ng/mL) |
| 53 | >85 | 2-4 PEG/SS1P | 2.36 |

### Example 3D

### Preparing Releasable BCN3-3 MONO-12K/20K/30K BCN3-SS1P

In PEGylation reactions employing releasable linkers such as BCN3-mono-12k/20k/30k-NHS, anti-mesothelin immunotoxin SS1P at 1.5-2.5 mg/mL was PEGylated in 0.05-0.1 M sodium phosphate, pH 7.6-7.8, 25° C. With fast stirring without creating foam, the PEG powder at 40:1-50:1 reaction molar ratio of PEG to SS1P was added to SS1P solution at 1 g/min rate. Alternatively, the PEG powder was predissolved in 1 mM HCl and added to the SS1P solution with stirring. The reaction was continued at 25° C. for 1 hour and quenched by adding glycine or by lowering pH to 6.5 with sodium phosphate, mono basic. The conjugate was purified on size exclusion column (Superdex 200 or 75 Hiload, Amersham, N.J.) equilibrated in 20 mM sodium phosphate, pH 6.5, 140 mM NaCl. Alternatively, the conjugate was purified on anion exchange column (Q Sepharose Fast Flow column, Amersham, N.J.) using 10 mM sodium phosphate, pH 7.4 as an equilibrium buffer and 0.3 M sodium chloride in 10 mM sodium phosphate, pH 7.4 as a gradient elution buffer or 10 mM Tris, pH 7.6 as an equilibrium buffer and 0.3 M sodium chloride in 10 mM Tris, pH 7.6 as a gradient elution buffer. The fractions containing BCN3-mono-SS1 were identified on precast 4-20% SDS non-reducing gel (Invitrogen, Calf.), concentrated using Centriplus 30k (Millipore, Mass.), and passed through a sterile filter (Acrodisc Syringe Filter, 0.2 μm HT Tuffyn membrane, Pall, Mich.). The protein concentration was determined by bicinchoninic acid assay (Pierce, Ill.) using bovine serum albumin as a standard and the cytotoxicity was analyzed on A431/K5 cells. The number of PEG polymers per SS1P molecule was estimated by SDS-PAGE (precast 4-20% SDS non-reducing gel, Invitrogen, CA). The purity of the product was analyzed on a TSK gel filtration column equilibrated in 50 mM sodium phosphate, pH6.5, 150 mM NaCl (G4000SWXXL, 7.8×30 cm, 8 μm, Tosoh Biosciences). The peak area was calculated at 220 nm.

### Example 3E

### Preparation of Releasable BCN3-mono-30K-SS1P

With fast stirring, 239-mg BCN3-mono-30k-NHS (Enzon, E1245-160A) was added to 5.9-mL of 1.7 mg/mL SS1P in 0.1 M sodium phosphate, pH 7.8. The reaction was continued at 25° C. for 60 min and the conjugate was purified on Q column (4-mL bed volume, 1×5.3 cm). The column equilibrium buffer was 10 mM Tris, pH 7.4 and the elution buffer contained 0.3 M NaCl in 10 mM Tris, pH 7.4. The fractions with the product peak were combined, concentrated on Centriplus 30k, and filtered through 0.2 μm sterile filter.

TABLE 4
Analysis of releasable BCN3-mono-30k-SS1P

| | Purity | | |
|---|---|---|---|
| Overall yield (%) | (% major peak area) | Composition | IC₅₀ (ng/mL) |
| 50 | >90 | 4-6 | 16.5 |
| | | PEG/SS1P | |

### Example 3F

### Preparation of Releasable RNL-8A-SS1P

15-mg of RNL-8a-12k-NHS (Enzon, E929-60A) was added to 2-mL of 1.94 mg/mL SS1P in 0.1 M sodium phosphate, pH 7.6 (20:1 reaction molar ratio). The reaction was continued at 25° C. for 60 min and quenched with glycine at 10:1 molar ratio of glycine to PEG. The conjugate was purified on a Superdex 200 Hiload column equilibrated in 20 mM sodium phosphate, pH 6.5, 140 mM NaCl. The fractions with the product peak were combined and filtered through 0.2 μm sterile filter.

TABLE 6
Analysis of releasable RNL-8a-SS1P

| Overall | | | IC₅₀ after 4 hrs at pH 8.5, |
|---|---|---|---|
| yield (%) | Composition | IC₅₀ (ng/mL) | 37° C. (ng/mL) |
| 60 | 4-6 PEG/SS1P | 251 | 4.5 |

### Example 3G

### Preparation of Hybrid DGA2-SC-SS1P

SS1P was modified first with the releasable DGA2-5k-NHS (Enzon, E1118-84) and then with permanent SC-12k-NHS (Enzon, V-05325). To 3-mL of 1.9 mg/mL SS1P in 0.1 M sodium phosphate, pH 7.6 solution was added 7-mg DGA2-5k-NHS powder. After 40-min reaction at 25° C., 11-mg SC-12k-NHS was added. The reaction molar ratio was DGA2:SC:SS1P=15:10:1. The reaction was continued for 60 min at 25° C. and then quenched with glycine (glycine:PEG=10:1). The hybrid conjugate was purified on a Superdex 200 Hiload column equilibrated in 20 mM sodium phosphate, pH 6.5, 140 mM NaCl. The combined fractions were concentrated on Centriplus 30k and passed through a sterile filter. The composition of the conjugate was 1-4 DGA2 and 1-3 SC per SS1P molecule.

TABLE 7
Analysis of Hybrid DGA2-SC-SS1P

| Overall yield | | IC₅₀ | IC₅₀ after 4 hrs at pH 8.5, 37° C. |
|---|---|---|---|
| (%) | Composition | (ng/mL) | (ng/mL) |
| 50 | 1-4 DGA2/1- | 1.78 | 1.04 |
| | 3 SC/SS1P | | |

### Example 4

### Preparing Non-Releasable PEG-SS1P Conjugates

For PEGylation reactions that employed permanent linkers, SS 1P at 1.5-2.5 mg/mL was PEGylated in 0.05 M sodium phosphate, pH 6.0-9.0, at 25° C. With fast stirring without creating foam, the PEG powder at 3:1 to 15:1 reaction molar ratio of PEG to SS1P was added to SS1P solution at 1 g/min. The reaction was continued at 25° C. for 1-2 hour for pH 7.4-9.0 reactions and 4 hours for pH 6.0-7.0 reactions. The reaction was quenched by adding glycine. The conjugate was purified by an anion exchange column (Q Fast Flow Sepharose, Amersham, N.J.) where the equilibration buffer was 10 mM Tris, pH 7.6 and the gradient elution buffer contained 0.3 M sodium chloride in 10 mM Tris, pH 7.6. If using size exclusion column (Superdex 200 or 75 Hiload (Amersham, N.J.), the equilibration buffer was PBS, pH 7.4. The sample was concentrated to about 1 mg/mL using Centriplus 30k (Millipore, Mass.) and passed through a sterile filter (Acrodisc Syringe Filter, 0.2 μm HT Tuffyn membrane, Pall, Mich.). The protein concentration was determined by bicinchoninic acid assay (Pierce, Ill.) using bovine serum albumin as a standard and the cytotoxicity was analyzed on A431/K5 cells. The ratio of PEG to SS1P molecule was estimated by SDS-PAGE (precast 4-20% SDS non-reducing gel, Invitrogen, Calf.). The purity of the product was analyzed on a TSK gel filtration column equilibrated in 50 mM sodium phosphate, pH6.5, 150 mM NaCl (G4000SWXXL, 7.8×30 cm, 8 μm, Tosoh Biosciences). The peak area was calculated at 220 nm.

### Example 4A

### Preparation of Mono PEG2-40K-SS1P at PH 7.8

The conjugate was formed by adding 63-mg PEG2-40k-NHS (Nektar, Calif.) to 7-mL of 1.5 mg/mL SS1P in 50 mM sodium phosphate, pH 7.8 solution with fast stirring (the reaction molar ration of PEG to SS1P was 10:1). The reaction was continued at 25° C. for 90 min and purified on a Q column (4-mL bed volume, 1×5.3 cm). The sample was diluted 6-fold with H₂O and loaded on the column which was equilibrated with 10 mM sodium phosphate, pH 7.8 and the conjugate was eluted with a linear gradient with 0.5 M NaCl in 10 mM sodium phosphate, pH 7.8 as the elution buffer. The compound was concentrated on Centriplus 30k to about 1 mg/mL and sterilized by passing through a sterile filter.

TABLE 8
Analysis of Mono PEG2-40k-SS1P

| Overall yield (%) | Composition | IC₅₀ (ng/mL) |
|---|---|---|
| 30 | 1 PEG/SS1P | 8.82 |

### Example 4B

### Preparation of Mono PEG2-20K-SS1P at PH 6.0

18-mg PEG2-20k-NHS (Nektar, Calif.) was added to 2-mL of 1.9 mg/mL in 0.1 M sodium phosphate, pH 6.0 solution with stirring, 25° C. (PEG:SS1P=15:1). The reaction was continued for 2 hours and mono PEG2-20k-SS1P was purified on a Mini Q-XL column (Amersham, N.J.) where the equilibration buffer was 10 mM sodium phosphate, pH 7.8 and the elution buffer contained 0.5 M NaCl in 10 mM sodium phosphate, pH 7.8.

TABLE 9
Analysis of mono PEG2-20k-SS1P

| Overall | | | |
|---|---|---|---|
| yield (%) | Composition | IC₅₀ (ng/mL) | IC₅₀ for Mock SS1P (ng/mL) |
| 20 | 1 PEG/SS1P | 26 | 0.5 |

### Example 4C

### Preparation of SC-SS1P at PH 6.5

The conjugate was formed at pH 6.5 and 15:1 reaction molar ratio (PEG:SS1P). With fast stirring without creating foam, 11-mg SC-12k-NHS (Enzon, V-05325) was added to 2.0 mL of 1.94 mg/mL SS1P in 0.1 M sodium phosphate, pH 6.5 solution. The reaction was continued at 25° C. for 120 min and quenched by adding glycine to a ratio of glycine:SS1P=10:1. The conjugate was purified by Q column or Superdex 200 Hiload column chromatography equilibrated as above. The fractions collected on Superdex 200 Hiload column in PBS, pH 7.4 contained 2-4 PEG per SS1P.

TABLE 10
Analysis of SC-SS1P

| Overall yield | | IC₅₀ | IC₅₀ after incubation with |
|---|---|---|---|
| (%) | Composition | (ng/mL) | hydroxylamine (ng/mL) |
| 50 | 2-4 | 19 | 9 |
| | PEG/SS1P | | |

### Example 4D

### Preparation of Alpha-Amino N-Terminal-PEG-SS1P

SS1P at 2 mg/mL was incubated with ALD-PEG-20k (Aldehyde-PEG-20k, Nektar, Calif.) at 10:1 reaction molar ratio (PEG:SS1P) in 100 mM sodium phosphate, pH 6.5, 25° C. for 3 h. The reduction of Schiff base was completed in 15 mM sodium cyanoborohydride at 25° C., 16 hours. Mono and Di PEG-SS1P were isolated on HiTrap Q-XL (Amersham Biosciences, N.J.) at pH 7.5. IC₅₀ was 39 ng/mL for mono and 310 ng/mL for di PEG-SS1P. SDS-PAGE showed that the PEG in mono PEG-SS1P was selectively located on the heavy chain (SS1-PE38VH) and the PEG in di PEG-SS1P was found on both heavy and light chains (VL) of SS1P.

### Example 4E

### Preparation of PEG-Hydrazide-SS1P

SS1P (1.5 mg/mL) was reacted with 250-mole fold of PEG-hydrazide-12k (Enzon, 929-12A) in the presence of 200-mole fold of 1-[3-(Dimethylamino) Propyl]-3-Ethyl Carbodiimide Hydrochloride (Aldrich, Wis.) in 1 mM HCl, pH 4.5, 25° C. for 1 hour. PEG-hydrazide-SS1P conjugate was isolated on HiTrap SP HP column (2×1 mL, Amersham, N.J.) with NaCl at pH 4.5.

TABLE 11
Analysis of PEG-Hydrazide-SS1P

| Overall yield (%) | Composition | IC₅₀ (ng/mL) |
|---|---|---|
| 37 | 1-4 PEG/SS1P | no inhibition |

### Example 5

### Analysis of Antigenicity of PEG-SS1P Conjugates

In order to obtain an estimate of the antigenicity of the prepared PEG-SS1P conjugates, the immunoreactivity of PEG-SS1P conjugates in antibody binding reactions were analyzed by Sandwich ELISA. The ELISA plate was coated with mouse monoclonal anti-PE40 antibody (NCI, DC) by incubating 200 ng antibody in 50 μL sodium bicarbonate on the plate at 25° C. for overnight. The plate was blocked next day with 250 μl/well of 1% BSA, 5% Sucrose, 0.05% NaN₃ in PBS, pH 7.4 for one hour at 25° C. and washed with wash buffer (PBS, pH 7.4, 0.05% Tween-20). 100-ng/50 μL/well SS1P or PEG-SS1P conjugate in diluent (0.1% BSA, 5% sucrose, 0.05% NaN₃, PBS, pH 7.4) was added for overnight incubation at 4° C. After washing with the wash buffer, 100-μL/well rabbit polyclonal anti-whole PE 794/2623 antibodies at 1:40,000 dilution (35-50% ammonium sulfate fraction, NCI, DC) was added. After 1.5 hour incubation at 25° C., the excess reagent was removed and the plate was washed with wash buffer. 50-μL horse radish peroxidase conjugated goat anti rabbit IgG at 1:5,000 dilution (Jackson, Pa.) was added. After 1 hour incubation at 25° C., the plate was washed three times with wash buffer and one time with H₂O. 100 μL TMB peroxidase substrate was added (Moss, Inc., PA) and the color development was stopped by adding 50 μL 1 M sulfuric acid in 15-20 min. Absorbance was read at 450 nm.

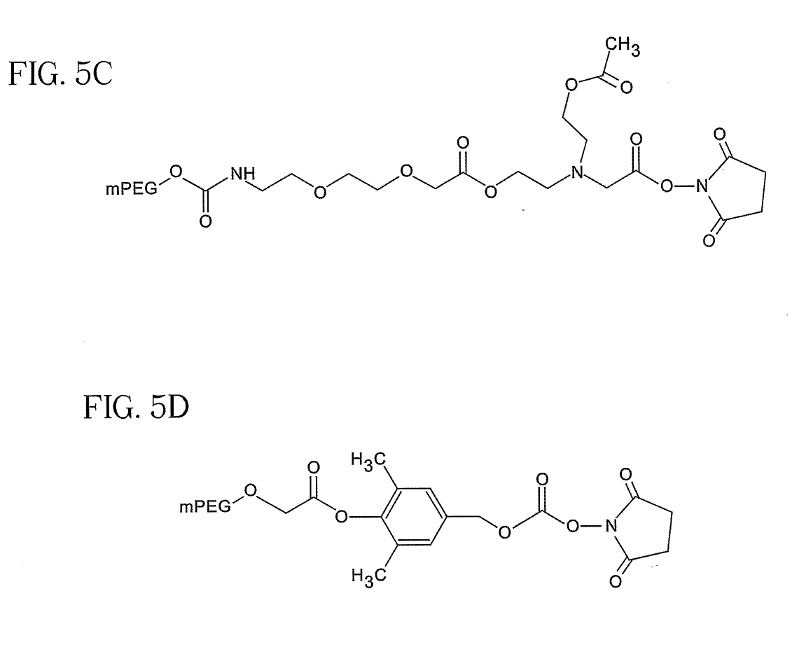

The absorbance readings (Y-axis) were plotted against concentration (ng) (X-axis) in FIG. 5. The results indicate that BCN3-mono-30K SS1P exhibited the lowest immunoreactivity and that unconjugated SS1P, exhibited the highest immunoreactivity. Reduced immunoreactivity to anti-SS1P antibodies is one of the desired traits sought from PEG-conjugates.

### Example 6

### Cytotoxicity Assay Confirming In Vitro Anti-Tumor Activity

In vitro anti-tumor activity was confirmed by a cytotoxicity assay, as follows.

1. A341K5 cells were grown in Dulbecco's modified Eagle's medium (“DMEM”) containing 10% FBS, 1× Penicillin/Streptomycin, 750 mg/ml G-418, and 200 mM L-glutamine at 37-C w/5% CO₂.

2. 2-fold serial dilutions of SS1P were prepared in 50 μl of the above medium in a 96 well flat bottom tissue culture plate. The SS1P concentration ranged from 50 ng/ml to 0.05 ng/ml. The A341/K5 cell control did not contain any SS1P.

3. 50 μl of the cell suspension (containing 2×10 cells) was dispensed in the above medium into each well of the tissue culture plate.

4. The plate was incubated at 37° C. w/5% CO₂ for 48 hours.

5. 15 μl of the MTT Dye Solution (Promega, Cat. No G4100) was added to each well and incubated at 37° C. w/5% CO₂ for 4 hours.

6. 70 μl of Solubilization/Stop Solution was added to each well and the plate was allowed to stand overnight.

7. The plate was recorded at 570 nm by VERSA MAX plate reader and the IC₅₀ was calculated using 4 parameter fit. The IC₅₀ values corresponded to 50% inhibition of cell growth. Native SS1P was included in assay sets for all comparisons to PEG-SS1P in IC₅₀ values from cytotoxicity assays.”

### Example 7

### Confirmation of In Vivo Anti-Tumor Activity

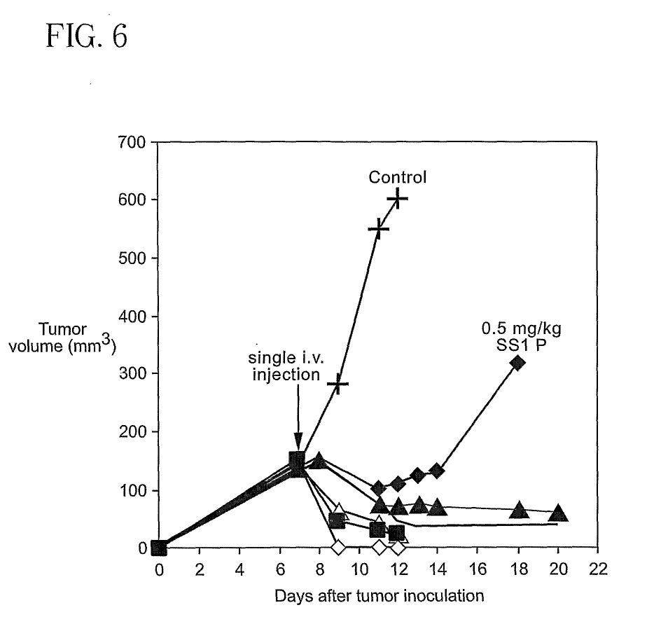

In vivo antitumor activities of PEG-SS1P conjugates were determined in mice bearing A431K5 tumors. 3×10⁶ A431K5 tumor cells were inoculated into nude mice (two mice per group) on day 0 and allowed to establish for 7 days. Starting on day 7, mice were treated with i.v. injections of 24k mPEG-BCN3—SS1P at 1.5, 2.0, 3.0, 4.0, and 6.0 mg/kg and SS1P at 0.5 mg/kg. The tumor was measured with a slide caliper (on the day indicated in FIG. 6) and the volume of the tumor was calculated as in Pai, L. H., Batra, J. K., FitzGerald, D. J., Willingham, M. C., and Pastan, I., 1991, Proc. Natl. Acad. Sci. USA, 88: 3358-3362.

As illustrated by FIG. 6, the mice treated with PEG-SS1P exhibited significantly decreased tumor volume, over a more prolonged period, in a dose-dependent manner.

### Example 8

### Comparison of In Vivo and In Vitro Anti-Tumor Activity

The methods employed for Examples 6 and 7, supra, were used to determine the effect of PEGylation on both in vitro cytotoxicity and on the in vivo antitumor activity of SS1P.

TABLE 12
Effect Of PEGylation On Cytotoxicity And Antitumor Activity Of SS1P

| | | | | | | | % tumor |
|---|---|---|---|---|---|---|---|
| | | | | | | | shrink on 7th |
| | | | | | | | day after 20 μg |
| | | | | | MTD | | or 40 μg/ |
| | | PEG# per | MW | | (μg/ | Cytotoxicity | mouse i.v. |
| No. | PEG-SS1P | SS1P* | (kDa)* | Linkage | mouse)** | (ng/mL) | injection |
| 1 | mPEG-12k- | 4-6 | ~130 | releasable, | 60 | 0.65 | 39 or 94 |
| | DGA2- | | | carbamate | | | |
| | RNL8a-SS1P | | | | | | |
| 2 | mPEG-24k- | ~3 | ~140 | releasable, | 60 | 2.04 | 27 or 70 |
| | BCN3-SS1P | | | amide | | | |
| 3 | mPEG-12k- | 4-6 | ~130 | releasable, | 80 | 251 | 50 or 63 |
| | RNL8a-SS1P | | | carbamate | | | |
| 4 | hybrid | 1-4, 1-3 | ~100 | releasable- | 40 | 1.78 | 65 or 61 |
| | DGA2-5k- | | | permanent, | | | |
| | SC-12k-SS1P | | | carbamate | | | |
| 5 | mPEG-30k- | 4-6 | ~200 | releasable, | 60 | 16.5 | 51 or 48 |
| | mono-SS1P | | | amide | | | |
| 6 | mPEG2-40k- | 1 | ~100 | permanent, | 60 | 8.8 | 53 or 64 |
| | SS1P | | | amide | | | |
| 7 | mPEG-12k- | 4-6 | ~130 | permanent, | 20 | no inhibition | 49 or one |
| | SC-SS1P | | | carbamate | | | died |

*MW and PEG # were estimated by SDS-PAGE;
**“MTD” is the maximum tolerated dose.

In Table 12, the cytotoxicity (mg/ml of each compound that results in an IC₅₀) provides an indication of the in vitro anti-tumor potency of each conjugate. The percentage of tumor shrinkage indicates the in vivo activity of each conjugate at the maximal tolerated dose. The artisan will note that there is not a complete correlation between the in vitro and in vivo activity. For example, compound No. 6 (mPEG2-40k-SS1P, a permanent linker) showed low in vitro inhibition of the tested tumor cells, but nevertheless provided a surprising degree of tumor shrinkage, in vivo. The IC50 value of 8.8 is approximately 3% of the IC50 value measured for native, nonconjugated SS1P. In addition, compounds 3-5 show a wide range of in vitro potencies, but all produce tumor shrinkage of about 50%. For all of these compounds, no significant tumor shrinkage was observed with two-fold increase in dosing. On the other hand, compound Nos. 1 and 2 showed distinct dose dependency, in vivo, implying specificity of treatment towards the tumor.

At day 7, non-conjugated SS1P (not shown by above table) resulted in only 3% shrinkage of the tumor volume, after a single dose of 10 μg/mouse (LD₁₀). The nonconjugated SS1P exhibited an in vitro IC₅₀ of 0.3 ng/ml. In contrast, the conjugated SS1P compounds provided significant tumor shrinkage.

### Example 9

### Further In Vivo Testing

Additional tests were conducted to further determine the toxicity and antitumor activity of releasable and permanent PEG-SS1P conjugates.

### A. Animal Toxicity Studies

The nonspecific toxicity of the PEGylated immunotoxins was examined in mice by intravenous administration of 2.0, 3.0, 4.0 or 6.0 mg/kg. Almost all of the deaths occurred within 4 days of treatment. Table 13, below, shows the toxicity data. The LD₅₀ of native SS1P was found to be about 1.0 mg/kg; by contrast, the PEGylated SS1P compounds were much better tolerated. For the anti-tumor studies we employed one or two dose levels below the dose that produced sickness or death.

TABLE 13

| | | DGA2- | Bicin3- | Bicin3- | RNL- | | | PEG2- |
|---|---|---|---|---|---|---|---|---|
| | SS1P | 12k | U-24k | mono-30k | 8a-12k | SC-12k | ALD-20k | 40k |
| Linker* | None | R | R | R | R | P | P | P |
| PEG mass on | None | 12 | 24 | 30 | 12 | 12 | 20 | 40 |
| linker (kDa) | | | | | | | | |
| PEG # | None | 4.2 | 2.6 | 3.9 | n.d. | n.d. | n.d. | 1.0 |
| (AFTC)^{†} | | | | | | | | |
| PEG # | None | 4, 5, 6 | 2, 3, 4 | n.d. | n.d. | n.d. | n.d. | n.d. |
| (CE) | | | | | | | | |
| PEG # | None | ~5 | ~3 | ~4 | ~5 | ~5 | 1 | 1 |
| (SEC) | | | | | | | | |
| PEG # | None | 4, 5, 6 | 3 | 4, 5 | 4, 5, 6 | 4, 5, 6 | 1 | 1 |
| (PAGE) | | | | | | | | |
| SS1P | n.a. | 125 | 90 | 140 | n.r. | n.r. | n.r. | n.r. |
| Release in vitro t_{1/2} | | | | | | | | |
| (hr)^{‡} | | | | | | | | |
| IC₅₀ (ng/ml) | 0.7 | 2.1 | 3.7 | 16.5 | >100 | >100 | 39 | 8.8 |
| LD₅₀ (mg | 1.0 | 3.0 | 3.0 | n.d. | 4.0 | n.d. | n.d. | n.d. |
| SS1P/kg) | | | | | | | | |
| K_{D} (nM)** | 0.78 | 39.5 | 8.1 | n.d. | n.d. | n.d. | n.d. | n.d. |
| Flow Cyt | 100 | 33 | 39 | 11 | 6 | 19 | 23 | 63 |
| (% of SS1P)^{††} | | | | | | | | |
| t_{1/2} (hr) | 0.44 | 4.8 | 4.8 | 5.0 | 4.4 | n.d. | n.d. | 2.5 |
| in vivo^{‡‡} | | | | | | | | |
| MRT (min) | 0.64 | 4.7 | 5.2 | 6.5 | 5.5 | n.d. | n.d. | 3.6 |
| AUC (min · mg/ml) | 0.36 | 28.6 | 31.3 | 18.8 | 0.77 | n.d. | n.d. | 2.0 |
| Tmax (min) | 30 | 120 | 120 | 180 | 60 | n.d. | n.d. | 10 |
| Cmax (μg/ml) | 3.4 | 51.5 | 47.0 | 20.9 | 1.1 | n.d. | n.d. | 8.7 |

Abbreviations for Table 13:
*R, releasable; P, permanent
“n.r.” is not released;
“n.a.” is not applicable,
“n.d.” is not determined.
^{†}PEG # is number of attached PEGs per SS1P determined by ammonium ferrothiocyanate (AFTC); capillary electrophoresis (CE); size exclusion chromatography (SEC); and SDS-PAGE
^{‡}half-life of SS1P release from PEG at pH 7.4, 25° C., in PBS
**K_{D} determined by Biacore; see text for discussion of kₒₙ and k_{off}
^{††}Flow cytometric analysis of % bound PEG-SS1P compound on A431-K5 cells; (native SS1P = 100% bound)
^{‡‡}pharmacokinetic parameters are t_{1/2}, biological half-life;
MRT, mean residence time;
AUC, area under the plasma concentration curve;
Tmax, time of maximal concentration;
Cmax, maximal concentration.

### B. Antitumor Activity of Releasable or Permanent PEG-SS1P

Antitumor activity of SS1P and PEG-SS1P compounds was determined in nude mice bearing A431-K5 human cancer cells that express mesothelin. Cells (3×10⁶) were injected s.c. into nude mice on day 0. Tumors˜140 mm³ in size developed in animals by day 7 after tumor implantation, after which animals were treated with i.v. injections of each of the immunotoxin compounds. In most experiments therapy with native SS1P or with the PEG-SS1P compounds and was given only once on day 7. In some experiments animals received PEG-SS1P twice on day 7 and 9 and SS1P three times on days 7, 9, and 11. The control groups received vehicle only. It was previously determined that SS1P inhibited tumor growth in a dose-dependent manner and that at least three doses of native SS1P (0.5 mg/kg) were required to achieve regressions in this mouse xenograft model. Usually three independent studies were conducted with each of the PEGylated compounds, using two different preparations of the SS1P conjugates.

As shown in Table 14, below, DGA2-12 kDa-PEG-SS1P (D-SS1P) was extremely active. A single dose of 2.0 mg/kg produced a 90% tumor shrinkage on day 14 and 2/4 mice showed complete tumor regressions. Mice receiving 2 doses of D-SS1P also showed a profound anti-tumor response with 4/8 mice showing a complete response and an average tumor size regression of 90%.

TABLE 14

| | | Day of | | | |
|---|---|---|---|---|---|
| | | best | % decrease | | |
| EXP # | Dose | response | in size | CR | Mice # |
| 17 | 1.0 mg/kg × 2 | 13 | 92% | 4/8 | 8 |
| 13, 14, | 2.0 mg/kg × 1 | 13 | 92% | 3/6 | 6 |
| 15-2 | | | (89% + 92% + | | |
| | | | 94%)/3 | | |
| 13 | Positive | 11 | 26% | n.a. | 2 |
| | control | | | | |
| | SS1P | | | | |
| | 0.5 mg/kg × 1 | | | | |
| 14, 15 | Negative | n.a. | n.a. | n.a. | 20 |
| 16, 17 | control | | | | |

As shown in Table 15, below, bicin3-U-24 kDa PEG-SS1P (B-SS1P) was also very active. Complete regressions were achieved in 1/4 mice receiving 2.0 mg/kg and 3/4 mice receiving 3.0 mg/kg. In mice receiving 2 doses of 2.0 mg/kg 4/8 mice showed complete regressions of the tumors.

TABLE 15

| | | Day of | % decrease | | |
|---|---|---|---|---|---|
| | | best | in | | |
| EXP # | Dose | response | size | CR | Mice # |
| 13, 14 | 2.0 mg/kg × 1 | 13 | 78% | 1/4 | 4 |
| 17 | 2.0 mg/kg × 2 | 13 | 92% | 4/8 | 8 |
| 14, 16 | 3.0 mg/kg × 1 | 11 | 92% | 3/4 | 4 |
| 13 | Positive control | 11 | 26% | n.a. | 2 |
| | SS1P | | | | |
| | 0.5 mg/kg × 1 | | | | |
| 14, 15 | Negative | n.a. | n.a. | n.a. | 20 |
| 16, 17 | control | | | | |

Table 16, below, shows the anti-tumor effects of the Bicin3-mono-30 kDa-PEG-SS1P; It appears to be somewhat less active than the first 2 compounds tested. At 3 mg/kg a single dose caused a maximal decrease in tumor size of 68% whereas 2 doses of 2.0 mg/kg caused an average decrease in size of 92% with 1/5 complete remissions.

TABLE 16

| | | Day of | % | | |
|---|---|---|---|---|---|
| | | Best | Decrease | | Mice/ |
| EXP # | Dose | response | in size | CR | group |
| 17 | 2.0 mg/kg × 2 | 13 | 68% | 1/5 | 5 |
| 15 | 3.0 mg/kg × 1 | 13 | 68% | 0/2 | 2 |
| 13 | Positive | 11 | 26% | n.a. | 2 |
| | control | | | | |
| | SS1P | | | | |
| | 0.5 mg/kg × 1 | | | | |
| 14 | Negative | n.a. | n.a. | n.a. | 20 |
| | control | | | | |

The other compound with a reversible linkage that showed significant anti-tumor activity is RNL-8a-12 kDa-PEG-SS1P. However as shown in Table 17, below, it is less active than the other compounds.

TABLE 17

| | | Day of | | | |
|---|---|---|---|---|---|
| | | best | % Decrease | | |
| EXP # | Dose | response | in size | CR | Mice # |
| 14 | 2.0 mg/kg × 1 | 14 | 73% | 0/2 | 2 |
| 14 | 4.0 mg/kg × 1 | 14 | 89% | 1/2 | 2 |
| 13 | Positive control | 11 | 26% | n.a. | 2 |
| | SS1P | | | | |
| | 0.5 mg/kg × 1 | | | | |
| 14, 15 | Negative | n.a. | n.a. | n.a. | 20 |
| 16, 17 | control | | | | |

Further anti-tumor investigations were carried out with permanent PEG derivatives, multi-PEGylated SC-12 kDa PEG-SS1P and mono-PEGylated PEG2-40 kDa PEG-SS1P. Only the latter compound produced sustained regressions with 1/4 mice showing a complete regression at a dose of 3.0 mg/kg (see Table 18, below).

TABLE 18

| | | Day of | | | |
|---|---|---|---|---|---|
| | | best | % Decrease in | | |
| EXP # | Dose | response | size | CR | Mice # |
| 15, 16 | 2.0 mg/kg × 1 | 12 | 64%(68% + | 0/4 | 4 |
| | | | 60%)/2 | | |
| 15, 16 | 3.0 mg/kg × 1 | 14 | 86%(78% + | 1/4 | 4 |
| | | | 94%)/2 | | |
| 13 | Positive | 11 | 26% | n.a. | 2 |
| | control | | | | |
| | SS1P | | | | |
| | 0.5 mg/kg × 1 | | | | |
| 14, 15 | Negative | n.a. | n.a. | n.a. | 20 |
| 16, 17 | control | | | | |

### C. Enhanced Permeability and Retention Effect

PEGylated proteins have been reported to accumulate in tumor xenografts via passive targeting by the enhanced permeability and retention (EPR) effect due to the leaky vasculature and deficient lymphatic drainage exhibited by tumors. To investigate the capability of non-targeted rPEGylated immunotoxins to demonstrate antitumor effects, Analogous rPEGylated compounds composed of a mutant of the CD22 targeted immunotoxin BL22 and the releasable PEGs, DGA2-12 kDa or bicin3-U-24 kDa were generated as summarized in Table 13, above. These compounds showed no anti-tumor activity, confirming that the anti-tumor activities of the PEGylated derivatives of SS1P are specific.

### D. Immunogenicity and Immunoreactivity of PEG-SS1P

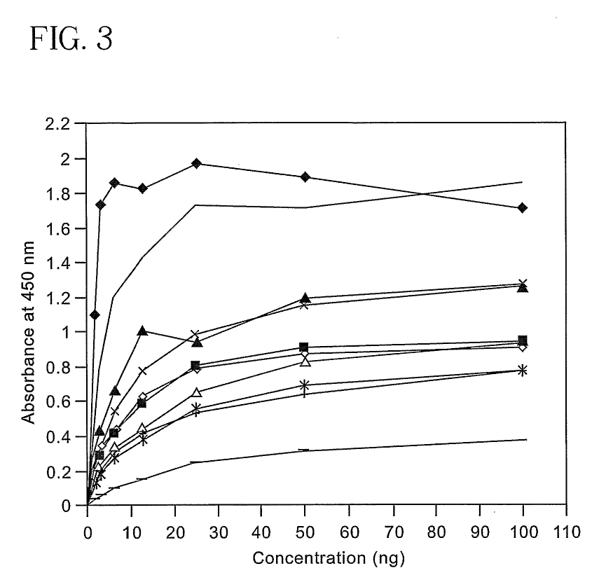

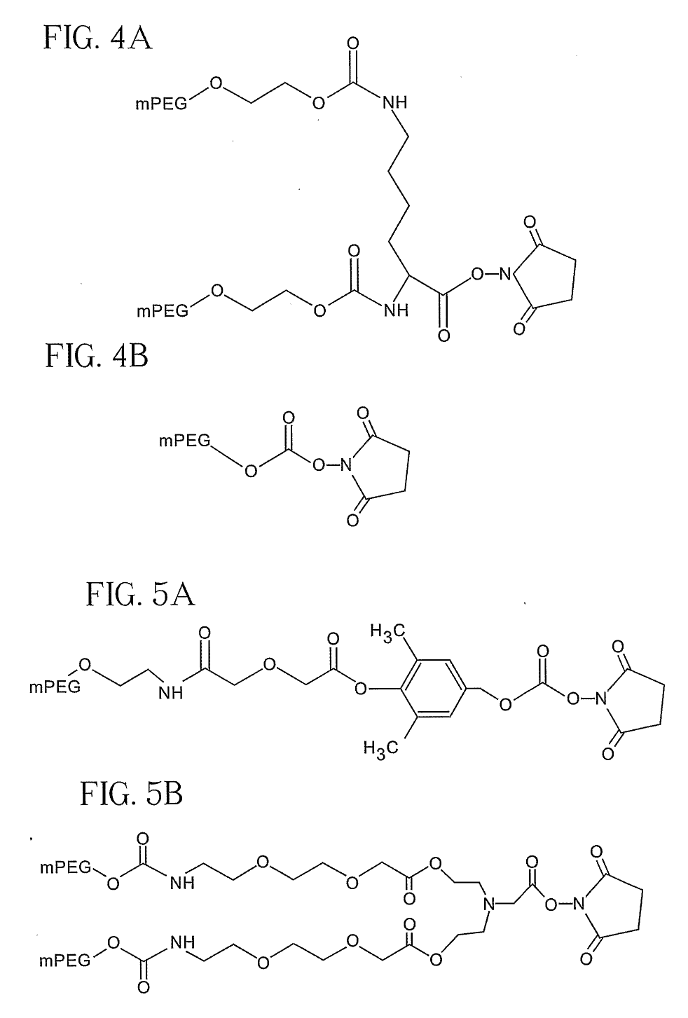

Since the IgG response of patients to administered immunotoxins may limit their duration of efficacy, the immunoreactivity of PEG-SS1P compounds toward anti-SS1P antibodies was assessed relative to native SS1P. Sandwich ELISA analysis of SS1P and several PEGylated SS1P derivatives in which plates coated with a mab reacting with PE38 were exposed to increasing amounts of each conjugate and the amount of immunotoxin bound detected with a polyclonal antibody to PE38 (see FIG. 3). Cross-reactivity of the PEG-SS1P compounds to the rabbit anti-PE antibodies is diminished compared to native SS1P. Two independent preparations of SS1P exhibited equivalent strong immunoreactivity, while the two independent preparations of heavily PEGylated bicin3-mono-30 kDa-PEG-SS1P demonstrated the weakest cross-reactivity. The N-terminally monoPEGylayed ALD-20 kDa-PEG-SS1P was strongly reactive with the anti-PE antibodies, as expected due to PEG attachment on the Fv portion of the Fv-PE toxin. Other rPEGylated derivatives were intermediate in signal. This suggests a possible strategy for evading the rapid clearance in patients due to existing neutralizing antibodies. To assess the immunogenicity of the PEG-SS1P compounds, BALB/c mice were immunized i.v., once per 7 days for four total doses, with SS1P and PEG-SS1P compounds at doses of 2.5 μg per mouse (SS1P), or 10 μg per mouse (PEG-SS1P), respectively. Higher doses of unmodified SS1P could not be administered due to toxicity of the native toxin. Blood samples were collected every 7 days, before the subsequent immunization. The specific IgG and IgM levels were determined by capture ELISA. At day 28, IgM antibody levels were similar for SS1P and two rPEGylated derivatives; IgG antibody levels were also similar at day 28 for these compounds (data not shown). The mouse antisera were also investigated in cytotoxicity assays, and both the anti-SS1P and anti-PEG-SS1P antisera were determined to contain neutralizing antibodies.

Although immunogenicity in mice does not predict human immunogenicity, these data do suggest that an immune response is not precluded in the current rPEGylated SS1P compounds. The great increase in blood residency time of the PEG-SS1P compounds in mice might provide a counteracting opportunity for development of an immune response. To assess the cross-reactivity of PEG-SS1P and native SS1P to human antibodies versus SS1P, antiserum were collected from two patients undergoing SS1P therapy and analyzed in competition ELISA, (data not shown). Human antiserum was mixed with dilutions of either SS1P or PEG-SS1P, and then added to SS1P coated plates. The detection reagent was rabbit anti-human IgG (HRP conjugate). The releasable or permanent PEG-SS1P compounds demonstrated cross-reactivity to the anti-SS1P human antisera, although the PEG-SS1P compounds exhibited a one- to two-log reduced binding efficiency in this competition immunoassay when compared to native SS1P. These data suggest that PEGylated SS1P may be less prone to rapid clearance in patients with existing antibodies to the native SS1P protein.

### E. Target Binding and Uptake of PEG-SS1P.

To assess the bioactivity of the PEG-SS1P derivatives in (1) mesothelin binding, (2) cell surface mesothelin binding, (3) cell uptake and processing, we performed several studies. For the DGA2-12 kDa-PEG-SS1P and bicin3-U-24 kDa-PEG-SS1P compounds, a determination of K_{D}, kₒₙ and k_{off} were performed by Biacore analysis. The high affinity SS1P (K_{D}=0.7 nm) demonstrated diminished affinity in the DGA2-12 (K_{D}=39.5 nM) and bicin-U-24 (K_{D}=8.1 nM) derivatives. For SS1P, kₒₙ=1.1×10⁶ M⁻¹sec⁻¹, while the DGA2-12 and bicin3-U-24 derivatives have kₒₙ rates of 6.3×10³ M⁻¹sec⁻¹ and 6.4×10⁴ M⁻¹sec⁻¹, respectively. Dissociation rates (k_{off}) were relatively conserved among the compounds, SS1P (8.3×10⁻⁴ sec⁻¹), DGA2-12 kDa-PEG-SS1P (2.5×10⁻⁴ sec⁻¹), and bicin3-U-24 kDa-PEG-SS1P (5.1×10⁻⁴ sec⁻¹). Flow cytometry investigations were conducted with the PEG-SS1P compounds to evaluate their cell surface binding to A431-K5 cells. SS1P and PEG-SS1P compounds at equimolarity were incubated with the cells, and mouse anti-PE-40 mAb was added, followed by phycoerythrin-conjugated goat anti mouse polyclonal antibody. As seen in Table 13, above, a qualitative correlation is observed for the PEG-SS1P compounds between higher efficiency of cell target binding, with greater potency in cytotoxicity assays.

1. A polymer-conjugate of SS1P comprising SS1P covalently attached to a substantially non-antigenic polymer.

2. The polymer-conjugated SS1P of claim 1, wherein the SS1P is releasable or nonreleasable in vivo from the substantially non-antigenic polymer.

3. The polymer-conjugated SS1P of claim 1, wherein the substantially non-antigenic polymer is a polyalkylene oxide.

4. The polymer-conjugated SS1P of claim 3, wherein the polyalkylene oxide is polyethylene glycol.

5. The polymer-conjugated SS1P of claim 4 that is selected from the group consisting of mPEG-12k-DGA2-RNL8a-SS1P, mPEG-24k-BCN3—SS1P, mPEG-12k-BCN3-mono-SS1P mPEG-12k-RNL8a-SS1P, mono mPEG2-40k-SS1P, mPEG-12k-SC-SS1P mPEG-12k-hydrazide-SS1P, mono mPEG-20k-Ald-SS1P and di mPEG-20k-Ald-SS1P.

6. The polyethylene glycol SS1P protein conjugate of claim 1, wherein the substantially non-antigenic polymer ranges in size from about 15 kDa to about 50 kDa.

7. The polymer-conjugated SS1P of claim 1, comprising a number of PEG chains ranging from 1 to 4.

8. The polymer-conjugated SS1P of claim 1, wherein SS1P comprises a disulfide linked dimer, the dimer comprising a polypeptide of SEQ ID NO: 5 and a polypeptide of SEQ ID NO: 7.

9. A pharmaceutical composition comprising the polymer-conjugated SS1P of claim of claim 1.

10. The pharmaceutical composition of claim 9, further comprising a second anti-cancer agent.

11. A method of treating a tumor or cancer in an animal comprising administering an effective amount of the polymer-conjugated SS1P of claim 1 to the animal, wherein the tumor or cancer has the property of expressing a mesothelin antigen.

12. The method of claim 11 wherein the tumor or cancer is a type selected from the group consisting of a mesothelioma, an ovarian cancer, a squamous cell carcinoma and a pancreatic adenocarcinoma.

13. The method of claim 11, that comprises administering at least one additional anticancer agent together with the polymer-conjugated SS1P.

14. The method of claim 11, that comprises administering at least one additional anticancer agent before or after administering the polymer-conjugated SS1P.

15. A polymer conjugate of the formula:
(R₁)_{z}—NH—(ITX) (I)
wherein
(ITX) represents the immunotoxin, or a derivative or fragment thereof;
NH— is an amino group of an amino acid found on the ITX, derivative or fragment thereof for attachment to the polymer;
z is a positive integer, of from about 1 to about 6; and
R₁ is a substantially non-antigenic polymer residue that is attached to the ITX in a releasable or non-releasable form.


SEQUENCE LISTING (8 sequences)

SEQ ID NO: 1 - DNA, 3647 bp, Artificial Sequence
  synthetic nucleotide sequence
taatacgactcactatagggagaccacaacggtttccctctagaaataattttgtttaactttaagaaggagatatacatatggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggtccaagcaccctctcacgttcggttgcgggacaaagttggaaataaaataatgaattcggctgctaacaaagcccgaaaggaagctgagttggctgctgccaccgctgagcaataactagcataaccccttgggcctctaaacgggtcttgaggggttttttgctgaaaggaggaactatatccggatcggagatcaattctggcgtaatagcgaagaggcccgcaccgatcgcccttcccaacagttgcgtagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaactgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttggggatcctctagagttgcatgcctgcaggtccgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggagttccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccactttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggactctagaggatccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctggtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcttgagatctcgatccgcgaaat

SEQ ID NO: 2 - DNA, 4833 bp, Artificial Sequence
  synthetic nucleotide sequence
taatacgactcactatagggagaccacaacggtttccctctagaaataattttgtttaactttaagaaggagatatacatatgcaggtacaactgcagcagtctgggcctgagctggagaagcctggcgcttcagtgaagatatcctgcaaggcatctggttactcattcactggctacaccatgaactgggtgaagcagagtcatggaaagtgccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaaaagcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaactgccgcgaccggccggctcccttcgcaggagccggccttctcggggcctggccatacatcaggttttcctgatgccagcccaatcgaatatgaattcggctgctaacaaagcccgaaaggaagctgagttggctgctgccaccgctgagcaataactagcataaccccttgggcctctaaacgggtcttgaggggttttttgctgaaaggaggaactatatccggatcggagatcaattctggcgtaatagcgaagaggcccgcaccgatcgcccttcccaacagttgcgtagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttggggatcctctagagttgcatgcctgcaggtccgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggagttccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccactttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggactctagaggatccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcggtagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgactgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgcaaggagatggcgcccaacagtcccccggccacggggcctgctcaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcttgagatctcgatccgcgaaat

SEQ ID NO: 3 - protein, 9 aa, Artificial Sequence
  synthetic peptide
QQWSGYPLT

SEQ ID NO: 4 - protein, 9 aa, Artificial Sequence
  synthetic peptide
QQWSKHPLT

SEQ ID NO: 5 - protein, 472 aa, Artificial Sequence
  synthetic polypeptide
MQVQLQQSGPELEKPGASVKISCKASGYSFTGYTMNWVKQSHGKCLEWIGLITPYNGASSYNQKFRGKATLTVDKSSSTAYMDLLSLTSEDSAVYFCARGGYDGRGFDYWGQGTTVTVSSKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 6 - DNA, 1419 bp, Artificial Sequence
  synthetic polypeptide
atgcaggtacaactgcagcagtctgggcctgagctggagaagcctggcgcttcagtgaagatatcctgcaaggcatctggttactcattcactggctacaccatgaactgggtgaagcagagtcatggaaagtgccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaaaagcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaagcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaa

SEQ ID NO: 7 - protein, 107 aa, Artificial Sequence
  synthetic polypeptide
MDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGCGTKLEIK

SEQ ID NO: 8 - DNA, 321 bp, Artificial Sequence
  synthetic nucleotide sequence
atggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggtccaagcaccctctcacgttcggttgcgggacaaagttggaaataaaa